(19) United States
(12) Patent Application Publication
(10) Pub. No.: US 2024/0082165 A1
(43) Pub. Date: Mar. 14, 2024

(54) METHODS AND COMPOSITIONS FOR PRECISION RELEASE OF PROBIOTICS TO IMPROVE HUMAN HEALTH

(71) Applicants: Rajat DHIR, Venice, CA (US); Gregor REID, Venice, CA (US); Ara KATZ, Venice, CA (US); SEED HEALTH INC., Venice, CA (US)

(72) Inventors: Rajat DHIR, Venice, CA (US); Gregor REID, Venice, CA (US); Ara KATZ, Venice, CA (US)

(21) Appl. No.: 18/262,870

(22) PCT Filed: Jan. 26, 2021
(86) PCT No.: PCT/US21/15107

§ 371 Date: Jul. 25, 2023

(51) Int. Cl. A61K 9/48; A61K 31/37; A61K 35/744

(52) U.S. Cl. CPC A61K 9/4808; A61K 31/37; A61K 35/744

(57) ABSTRACT

Synbiotic compositions including both a prebiotic component and a probiotic component are provided. The prebiotic component includes at least one punicalagin, and the probiotic component includes a rationally defined and assembled consortium of microbial strains. Delivery capsules for oral administration of the synbiotic compositions and methods of using the synbiotic compositions to treat disease are also provided.

### FIELD OF THE INVENTION

The present invention is related generally to microbial biologics, and particularly to rationally defined microbial consortia that are effective to impart health across organ systems by modulating the function of the naive gut microbiota and host tissue.

### BACKGROUND OF THE INVENTION

Microbial biologics have emerged as promising therapies to impart beneficial function to resident gut microbial communities and host tissue. The administration of multi-strain, non-redundant microbial consortia are currently being clinically evaluated for the prevention and treatment of a range of conditions including food allergy, autism, and cancer, but the ability to reproducibly assemble and deliver complex consortia for prophylactic use remains nascent.

Humans are colonized by trillions of microorganisms in the gastrointestinal tract, oral cavity, skin, nares, and urogenital tract. Besides resident microbes, transient species constantly enter the body through the ingestion of foods, in particular those that are fermented. It has been over a century since the correlation between enhanced longevity and the consumption of yogurt containing lactic acid-producing bacteria was first observed, and since Bifidobacterium spp. were first detected in the feces of breastfed infants but not of those who were formula-fed and suffering from diarrhea. These early “bacteria for health” concepts have culminated in the widely adopted use of probiotics as “live microorganisms that, when administered in adequate amounts, confer a health benefit on the host”.

Probiotics historically encompassed the genera Bifidobacterium and Lactobacillus. Over 250 Lactobacillus species were identified, which appeared to be highly genetically distinct from one another but also metabolically, ecologically, and functionally diverse. These observations led to a recent reclassification of the genus Lactobacillus into 25 genera, including the emended genus Lactobacillus (which encompasses host-adapted organisms that are now referred to as the Lactobacillus delbrueckii group), Paralactobacillus, and 23 novel genera.

The National Institutes of Health Human Microbiome Project showed great variation in the diversity and abundance of microbes among healthy individuals, indicating that there is no universally healthy microbiome. Despite this variety of healthy states, important correlations between health and disease status and the presence or abundance of specific microbial species have been established, raising the possibility that manipulation of these communities can prevent and treat illnesses. This concept fostered the isolation of specific microbiota members from healthy individuals for the development of so-called live biotherapeutic products (LBPs). These added to the classic lactobacilli- and Bifidobacterium-based probiotic products, aiming to beneficially impact disease status. Notably, gut microbiota interventions not only bring health benefits to the local environment but also impact distantly, through for instance the gut-skin, gut-heart, and gut-brain axes.

Bacterial members of the microbiome can impact health and disease by at least four separate pathways: immunomodulation, pathogen inhibition, improved epithelial barrier integrity, and the production of beneficial metabolites or the removal of detrimental molecules.

Immunomodulation

The intestinal mucosae contain several specialized cell types involved in immunomodulation, each of which expresses different pattern recognition receptors (PRRs), including NOD-like and Toll-like receptors (TLRs). Bacteria express a range of mostly cell envelope-located and -secreted microorganism-associated molecular patterns (MAMPs). Upon exposure to MAMPs, PRRs respond by activating associated adaptor proteins that are linked to nuclear factor-κB and mitogen-activated protein kinase signaling cascades, resulting in altered expression levels of response genes, including those encoding chemokines, cytokines, and antimicrobial peptides. This importantly establishes the intestine as a key region for immunomodulation and therefore a primary target for bacterial strains that can create and restore metabolic homeostasis.

The availability of the first full genome sequences of lactobacilli marked the beginning of an era in which researchers unraveled the molecular mechanisms by which probiotic model strains communicate with the host immune system. In these model strains, MAMP-encoding genes were deleted to assess the impact on microbe-host communication. For example, removal and/or modification of lipoteichoic acid (LTA) from the cell envelope of Lactobacillus acidophilus NCFM, Lactiplantibacillus plantarum WCFS1, and Lacticaseibacillus rhamnosus GG resulted in strains with altered TLR-2 signaling cascades. The resulting cytokine profiles were distinctly strain-specific, potentially caused by differences in LTA chain length or substitution levels and/or types, but consistently more anti-inflammatory (more IL-10 and/or less IL-12). Moreover, the mutants improved disease symptoms compared to the parental strains in mouse colitis models. Similarly, peptidoglycan is present in all lactobacilli, but subtle structural variations play a pivotal role in probiotic efficacy. Lactobacillus acidophilus LS33 peptidoglycan contains a strain-specific muropeptide responsible for NOD-2 dependent IL-10 induction, and the synthesized ligand (M-tri-Lys) protected mice from colitis in a NOD2-dependent but MyD88-independent manner. Taken together, several conserved MAMP-PRR interactions have been unraveled at the molecular level, and their impact on the immune system translates to alleviation of disease symptoms in animal models. However, subtle structural differences in the bacterial polymers result in strain-specific efficacies that are not fully understood, thereby preventing bioinformatics-based prediction of optimal probiotic functionality.

Strain-specificity resulting from conserved MAMPs is further substantiated by the fact that large sets of MAMPs such as exopolysaccharides, wall teichoic acid, pili, and proteinaceous components, all established to impact host immune response, are not present in all probiotic strains.

Epithelial Barrier Integrity

A single-cell layer of intestinal epithelial cells linked together by tight junction proteins constitutes the gut barrier. This barrier regulates permeability, and compromise of this barrier can result in Gram-negative pathogens, lipopolysaccharides, or toxic metabolites entering the bloodstream from the gut, initiating an inflammatory cascade associated with necrotizing enterocolitis (NEC), inflammatory bowel disease (IBD), auto-immune disease, and metabolic disorders including diabetes and obesity. In addition to tight junctions, Paneth cells produce defensin to maintain sterile crypts, and goblet cells secret mucins. Select microbial strains have been shown to improve epithelial barrier function by upregulating signaling pathways that increase tight junction function, enhance defensin production, or attenuate apoptosis.

Metabolites

Another mechanism by which microbes impact host health is through the metabolites they produce in, or remove from, the gut environment. A clear-cut example is folate, which is not synthesized by mammals and must be obtained from exogenous sources. This B vitamin is a vital nutrient required for human metabolic pathways, including nucleic acid and amino acid synthesis, DNA methylation, and survival of regulatory T cells. Increased folate intake during pregnancy can decrease neural tube birth defects. Bifidobacterium strains produce folate, and daily supplementation of healthy volunteers with Bifidobacterium adolescentis SD-BA5-IT increases folate in feces, providing an important health benefit. Besides folate, other B vitamins such as thiamine and riboflavin are also produced by probiotic Bifidobacterium species and lactobacilli.

Gut-Skin Axis

The microbes residing on human skin are subjected to variable environmental pressures, including temperature, humidity, and pH, as well as antimicrobial peptides and lipids. Notably, skin structures such as hair follicles and sebaceous, eccrine, and apocrine glands represent distinct niches that harbor distinct microbiota. At least 19 phyla are known to be part of the healthy skin microbiota, with particularly abundant genera including Propionibacterium, Staphylococcus, and Corynebacterium. The gut microbiota can distally affect the skin in quantifiable ways via several intertwined mechanisms, e.g. immunomodulation, inhibition of pathogens, production of beneficial metabolites, and/or impacting the gut epithelial barrier. For instance, the integumentary system of healthy mice is beneficially altered by oral supplementation with Limosilactobacillus reuteri ATCC 6475, leading to increased folliculogenesis and dermal thickness, as well as a lower pH of the skin and enhanced sebocyte production. Moreover, healthy probiotic-fed mice displayed increased and decreased serum levels of the anti- and pro-inflammatory cytokines IL-10 and IL-17, respectively, whereas in IL-10-deficient mice the integumentary system was not improved, demonstrating an immune-based mechanism of action of Limosilactobacillus reuteri. IL-10-induced changes typically involve the induction of Foxp3 T_{reg} cells, which is in line with the fact that administration of purified Foxp3 cells derived from Limosilactobacillus reuteri-fed donors also results in the probiotic-induced changes to the integumentary system in recipient mice, even without exposure to probiotic cells.

Another mechanism by which gut microbiota can impact the skin is through the metabolites they produce, which can access the bloodstream and affect distant sites. Alternatively, the gut bacteria themselves might be able to enter the bloodstream, especially when the gut epithelial barrier integrity is disturbed. For example, psoriasis vulgaris patients suffer from gut dysbiosis, and there are indications that this promotes DNA translocation to the bloodstream. The gut dysbiosis observed encompasses an increased microbial diversity, as well as higher abundance of Faecalibacterium prausnitzii, which is counterintuitive given that this genus, dominant in the healthy gut microbiota, has strains that produce high levels of the beneficial short chain fatty acid (SCFA) butyrate as well as anti-inflammatory peptides. In atopic dermatitis studies, short chain fatty acids have also been proposed to inhibit growth of pathogenic skin microbes such as S. aureus, while cutaneous skin commensals such as S. epidermidis and C. acnes tolerate wider SCFA shifts. Subcutaneously injected or topically applied butyrate reduces the contact hypersensitivity reaction in hapten-sensitized mice. Moreover, the T_{reg}-specific factors Foxp3 and IL-10 appeared up-regulated upon this treatment, indicating that T_{reg} cells can be induced by short-chain fatty acids. A wide range of bacterial strains, including from the genera Eubacterium, Bacteroides, Propionibacterium, Prevotella, Bifidobacterium and Lactobacillus, produce substantial amounts of short chain fatty acids, and therefore the observed beneficial effects for skin health are likely not exclusive to Faecalibacterium prausnitzii.

The triggering of several signaling transduction pathways (e.g. JNK and IKKP/NF-1<0) leads to the production of inflammatory cytokines and chemokines such as tumor necrosis factor α, the most important inflammatory mediator of psoriasis pathogenesis. Many microbes, such as B. fragilis, have been reported to be able to regulate the balance of Th1/Th2 immune response by producing polysaccharides A and B. Another proposed link between the microbiome and psoriasis is that psoriasis may reflect an abnormal innate immune response to the skin microbiome, mainly driven by IL-23 and IL-17, rather than being an auto-immune disease. Using a mouse model of psoriasis, it has been demonstrated that antibiotics targeting Gram-negative and Gram-positive bacteria could ameliorate psoriasiform dermatitis by inhibiting the production of IL-17 and IL-22. IL-22-producing T cells seem to play a crucial role in the aggravation of skin inflammation.

Commensal gut flora can promote skin allostasis by influencing T cell differentiation in response to various immune stimuli. Oral administration of Lactobacillus casei DN-114 001 has been shown to impair differentiation of CD8+ T cells into cutaneous hypersensitivity effector cells and decrease their recruitment to the skin when exposed.

Th17 cells are abundant in both the skin and intestine, as both organs contact the external environment. These cells and their pro-inflammatory cytokines are thought to directly contribute to the pathogenesis of several chronic inflammatory dermatoses, including psoriasis, Behcet's disease, and contact hypersensitivity. The balance between Th17 effector cells and their counterpart regulatory T cells is greatly influenced by the intestinal microbiome. Th17 cells can be eliminated in the intestinal lumen, or they may acquire a regulatory phenotype with immunosuppressive characteristics (rTh17) that restricts pathogenicity.

Other molecules synthesized by gut bacteria with the potential to modify skin either directly or indirectly have recently been reviewed. These include γ-aminobutyric acid and tryptamine from Lactobacillus species that inhibit itch, and acetylcholine for enhanced barrier function. To this end, there are indications that acne patients have enhanced gut epithelial barrier integrity, which could be the reason for the observed presence of and high reactivity to blood lipopolysaccharide endotoxins. Consistent with this hypothesis, an upregulation of substance P-containing nerves and a strong expression of this neuropeptide is mutually seen in both acne vulgaris and intestinal dysbiosis. Substance P can trigger inflammatory signals that result in the increase of pro-inflammatory mediators (IL-1, IL-6, TNF-α, PPAR-γ) implicated in the pathogenesis of acne.

A high glycemic load promotes an increase in insulin/insulin-like growth factor (IGF-1) signaling. This is thought to induce increased cytoplasmic expression of the metabolic forkhead box transcription factor (FoxO1), a sensor of cell nutrition state. FoxO1 ultimately triggers mammalian target of rapamycin complex 1 (mTORC1), a governor of metabolism and cell proliferation, to mediate sebaceous gland hyperproliferation, lipogenesis, and hyperplasia of acroinfundibular keratinocytes, thereby contributing to the development of acne. Gut microbiota influence the pathophysiology of acne via crosstalk between intestinal commensal bacteria and the mTOR pathway. Metabolites produced by gut microbiota have been shown to regulate cell proliferation, lipid metabolism, and other metabolic functions mediated by the mTOR pathway. The mTOR pathway can in turn affect the composition of intestinal microbiota through regulation of the intestinal barrier. In cases of intestinal dysbiosis and disrupted gut barrier integrity, this bidirectional relationship can result in a positive feedback cycle of metabolic inflammation. Given the important role of mTORC1 in the pathogenesis of acne, this relationship serves as a mechanism by which the gut microbiome can influence acne pathophysiology.

Orally consumed probiotics have been successfully used to alleviate and/or prevent skin diseases, including psoriasis vulgaris, atopic dermatitis, and acne vulgaris, via the gut-skin axis.

Cardiovascular disease remains the worldwide leading cause of death, with risk factors including elevated cholesterol levels and high blood pressure. A gut link with hypertension has been demonstrated in rodent models, revealing increased permeability of gut epithelial barrier and inflammatory state, combined with a shift in gut microbial genera potentially associated with the pathophysiological and immune status of the gut and high blood pressure. The role of epithelial barrier integrity has also been suggested in a study in which mice were fed a high fat diet, leading to a marked increase in the presence of bacterial DNA in various tissues.

Comparison of the gut microbiome of individuals with atherosclerotic cardiovascular disease with healthy controls revealed an increased abundance of Enterobacteriaceae, including potential pathogens such as Escherichia coli, Klebsiella spp., and Enterobacter aerogenes. Moreover, Ruminococcus gnavus, a bacterium previously associated with inflammatory bowel diseases, was found at a relatively high level. In contrast, butyrate-producing bacteria, including Roseburia intestinalis and Faecalibacterium prausnitzii, were relatively depleted in the samples derived from the diseased population

Comparison of the gut microbiomes of atherosclerotic cardiovascular disease patients with other disease cohorts (type 2 diabetes, liver cirrhosis, and obesity) revealed common deviations from healthy controls, suggesting that a less fermentative and more inflammatory gut environment is a shared characteristic in several diseases. To this end, overweight individuals receiving propionate delivered directly in the colon have already been shown to display reduced energy intake, adiposity, and lipid liver content, and increased plasma levels of peptide YY (PYY) and glucagon-like peptide 1 (GLP-1) produced locally by enteroendocrine L cells.

The healthy gut microbiome also has the capacity to dehydrogenate cholesterol to coprostanol, which is poorly absorbed. The enzymatic activity for this conversion is encoded by ismA in a clade of uncultured microorganisms prevalent in geographically diverse human cohorts. Individuals harboring coprostanol-forming microbes have lower fecal cholesterol levels and lower serum total cholesterol, with effects comparable to those attributed to variations in lipid homeostasis genes. This suggests a direct link between one specific functionality in the microbiome and human health.

Although probiotics do not harbor ismA homologues, a double-blind, placebo-controlled, randomized study in which healthy, normal to mildly hypercholesterolemic adults received supplementation with L. plantarum ECGC 13110402 revealed a reduction in low-density lipoproteins, triglycerides, total cholesterol, and systolic blood pressure, and an increase in high-density lipoproteins within 12 weeks. Primary bile acids are synthesized in the liver from cholesterol, and L. plantarum ECGC 13110402 produces high levels of bile salt hydrolase. The exact mechanism remains unknown, but the net effect of this enzymatic activity is lower cholesterol levels, including low-density lipoprotein, in humans and animal models. Moreover, the strain displayed the ability to reduce cholesterol in vitro which implies a complementary mechanism of action, for instance the capacity to internalize cholesterol.

### SUMMARY OF THE INVENTION

In one aspect of the present invention, a swallowable capsule for enteral administration of a synbiotic composition comprises an inner capsule, comprising a probiotic component of the synbiotic composition, the probiotic component comprising a consortium of microbial strains; and an outer capsule, surrounding and enclosing the inner capsule, comprising a prebiotic component of the synbiotic composition, wherein the outer capsule is configured to be substantially completely destroyed or dissolved after three hours in the environment of the human stomach and small intestine, wherein the inner and outer capsules are configured such that a proportion of cells in the consortium of microbial strains that remain viable after three hours in the environment of the human stomach and small intestine is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, and wherein the inner capsule is configured, upon entry into the colon of a human subject to whom the swallowable capsule is administered, to release at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of viable cells of the consortium of microbial strains into the colon.

In embodiments, the capsule may comprise the prebiotic component in an amount of from about 1 mg to about 400 mg, or from about 25 mg to about 375 mg, or from about 50 mg to about 350 mg, or from about 75 mg to about 325 mg, or from about 100 mg to about 300 mg, or from about 125 mg to about 275 mg, or from about 150 mg to about 250 mg, or from about 175 mg to about 225 mg, or about 200 mg.

In embodiments, the capsule may comprise the consortium of microbial strains in an amount of from about 62.5 million AFU to about 312.5 billion AFU, from about 625 million AFU to about 250 billion AFU, from about 1.25 billion AFU to about 125 billion AFU, from about 6.25 billion AFU to about 62.5 billion AFU, from about 12.5 billion AFU to about 50 billion AFU, from about 18.75 billion AFU to about 37.5 billion, or from about 25 billion AFU to about 31.25 billion AFU.

In embodiments, the inner capsule may further comprise a pharmaceutically acceptable vehicle for the probiotic component.

In embodiments, the outer capsule may further comprise a pharmaceutically acceptable vehicle for the prebiotic component.

In embodiments, the capsule may be substantially free of any liquid vehicle or solvent.

In embodiments, the prebiotic component may comprise at least one compound that can be converted, by a microbial strain present in the healthy human gut microbiota, into a bioactive metabolite, which may in some embodiments be a punicalagin. The at least one punicalagin may, but need not, be derived or extracted from at least one pomegranate. The prebiotic component may, but need not, further comprise at least one additional compound derived or extracted from at least one pomegranate. The prebiotic component may, but need not, consist essentially of a polyphenolic pomegranate derivative or extract comprising the at least one punicalagin. The at least one punicalagin may, but need not, be capable of being metabolized, by at least one bacterial strain known to inhabit the human gastrointestinal tract, into a urolithin. The at least one punicalagin may, but need not, be capable of being metabolized, by at least one microbial strain of the consortium of the probiotic component, into a urolithin. The urolithin may, but need not, be urolithin-A.

In embodiments, the consortium of microbial strains may comprise at least two of (i) one or more digestive outcome-, gastrointestinal outcome-, or gut barrier function-improving microbial strains selected from the group consisting of Bifidobacterium breve SD-BR3-IT, Lactiplantibacillus plantarum SD-LP1-IT, Bifidobacterium longum SD-BB536-JP, Bifidobacterium infantis SD-M63-JP, Lacticaseibacillus rhamnosus HRVD113-US, Bifidobacterium lactis HRVD524-US (B1-04), Bifidobacterium breve HRVD521-US, Lacticaseibacillus casei HRVD300-US, Bifidobacterium longum HRVD90b-US, Bifidobacterium lactis SD150-BE, Lacticaseibacillus rhamnosus SD-GG-BE, Limosilactobacillus reuteri RD830-FR, Lactobacillus crispatus SD-LCR01-IT, Limosilactobacillus fermentum SD-LF8-IT, Bifidobacterium lactis SD-BS5-IT, and Lacticaseibacillus rhamnosus SD-LR6-IT; (ii) one or more dermatological outcome-improving microbial strains selected from the group consisting of Ligilactobacillus salivarius SD-LS1-IT, Bifidobacterium longum SD-CECT7347-SP, Lacticaseibacillus casei SD-CECT9104-SP, and Bifidobacterium lactis SD-CECT8145-SP; (iii) one or more cardiovascular outcome-improving microbial strains selected from the group consisting of Lactiplantibacillus plantarum SD-LPLDL-UK and Bifidobacterium lactis SD-MB2409-IT; and (iv) one or more micronutrient-synthesizing microbial strains selected from the group consisting of Limosilactobacillus reuteri SD-LRE2-IT and Bifidobacterium adolescentis SD-BA5-IT. The consortium may, but need not, comprise at least three of (i) through (iv). The consortium may, but need not, comprise all four of (i) through (iv). The consortium may, but need not, comprise at least two of the digestive outcome-, gastrointestinal outcome-, or gut barrier function-improving microbial strains of (i). The consortium may, but need not, comprise all of the digestive outcome-, gastrointestinal outcome-, or gut barrier function-improving microbial strains of (i), all of the dermatological outcome-improving microbial strains of (ii), all of the cardiovascular outcome-improving strains of (iii), and all of the micronutrient-synthesizing strains of (iv). The consortium may, but need not, consist essentially of all of the digestive outcome-, gastrointestinal outcome-, or gut barrier function-improving microbial strains of (i), all of the dermatological outcome-improving microbial strains of (ii), all of the cardiovascular outcome-improving strains of (iii), and all of the micronutrient-synthesizing strains of (iv).

The advantages of the present invention will be apparent from the disclosure contained herein.

As used herein, “at least one,” “one or more,” and “and/or” are open-ended expressions that are both conjunctive and disjunctive in operation. For example, each of the expressions “at least one of A, B, and C,” “at least one of A, B, or C,” one or more of A, B, and C,” “one or more of A, B, or C,” and “A, B, and/or C” means A alone, B alone, C alone, A and B together, A and C together, B and C together, or A, B, and C together.

It is to be noted that the term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The embodiments and configurations described herein are neither complete nor exhaustive. As will be appreciated, other embodiments of the invention are possible utilizing, alone or in combination, one or more of the features set forth above or described in detail below.

### BRIEF DESCRIPTION OF THE DRAWINGS

FIG. 1A is an assembled view of a delivery capsule of the present invention.

FIG. 1B is an exploded view of a delivery capsule of the present invention.

FIG. 2 is a flowchart of a method for identifying and validating inhibitor microorganism consortia or components thereof.

FIGS. 3A and 3B are illustrations of gst-4::GFP expression in two separate untreated C. elegans replicates.

FIGS. 4A and 4B are illustrations of gst-4::GFP expression in two replicates of C. elegans treated with synbiotic compositions of the present invention.

FIG. 5 is an image of a western blot indicating claudin-1 expression in human epithelial cells treated with a synbiotic composition of the present invention.

FIGS. 6A and 6B illustrate bromocresol purple assays of microbial strains of synbiotic compositions of the present invention tested on liquid media and solid media, respectively.

FIG. 6C is a graph of visible light absorbance measurements of the microbial strains illustrated in FIGS. 6A and 6B.

FIG. 7 illustrates a simulated human intestinal microbial ecosystem.

FIG. 8 illustrates the number of viable bacterial counts of microbial strains of synbiotic compositions of the present invention in a simulated human intestinal microbial ecosystem.

FIG. 9 illustrates the change in pH over 48 hours in five simulated human intestinal microbial ecosystems.

FIG. 10 illustrates the change in butyrate concentration over 48 hours in five simulated human intestinal microbial ecosystems.

FIG. 11 illustrates the change in propionate concentration over 48 hours in five simulated human intestinal microbial ecosystems.

FIG. 12 illustrates the change in acetate concentration over 48 hours in five simulated human intestinal microbial ecosystems.

FIG. 13 illustrates the change in total short-chain fatty acid concentration over 48 hours in five simulated human intestinal microbial ecosystems.

FIGS. 14A and 14B are illustrations of the viability of microbial strains of synbiotic compositions of the present invention in a simulated human intestinal microbial ecosystem.

FIGS. 14C and 14D are illustrations of the viability of microbial strains of a prior art probiotic composition in a simulated human intestinal microbial ecosystem.

FIG. 15 is an illustration of histamine metabolism by microbial strains of synbiotic compositions of the present invention, relative to L. reuteri and a control.

FIGS. 16A and 16B are graphs of viable cell counts and water stability, respectively, of both a capsule of the convention and a liquid glycerol solvent solution, each containing the synbiotic composition of the invention, under a low-temperature, low-humidity condition.

FIGS. 17A and 17B are graphs of viable cell counts and water stability, respectively, of both a capsule of the invention and a liquid glycerol solvent solution, each containing the synbiotic composition of the invention, under a high-temperature, high-humidity condition.

FIG. 18A is a graph of viable cell counts of a capsule of the invention containing the synbiotic composition of the invention stored in a jar under accelerated degradation conditions.

FIG. 18B is a graph of viable cell counts of a capsule of the invention containing the synbiotic composition of the invention stored in a pouch under accelerated degradation conditions.

FIG. 19A is a graph of viable cell counts of a capsule of the invention containing the synbiotic composition of the invention stored in a jar under accelerated degradation conditions.

FIG. 19B is a graph of viable cell counts of a capsule of the invention containing the synbiotic composition of the invention stored in a pouch under accelerated degradation conditions.

FIGS. 20A and 20B are graphs of viable cell counts of capsules of the invention containing the synbiotic composition of the invention stored in a jar and a pouch, respectively, under typical long-term storage conditions.

### DETAILED DESCRIPTION OF THE INVENTION

Unless defined otherwise, all technical and scientific terms used herein have the same meaning as is commonly understood by one of ordinary skill in the art. All patents, applications, published applications, and other publications to which reference is made herein are incorporated by reference in their entirety. In the event that there is a plurality of definitions for a term herein, the definition provided in the Summary of the Invention prevails unless otherwise stated.

“CRISPR” (Clustered Regularly Interspaced Short Palindromic Repeats) loci refers to certain genetic loci encoding components of DNA cleavage systems, for example, used by bacterial and archaeal cells to destroy foreign DNA. A CRISPR locus can consist of a CRISPR array, comprising short direct repeats (CRISPR repeats) separated by short variable DNA sequences (called spacers), which can be flanked by diverse Cas (CRISPR-associated) genes. The CRISPR-Cas system, an example of a pathway that was unknown to science prior to the DNA sequencing era, is now understood to confer bacteria and archaea with acquired immunity against phage and viruses. Intensive research over the past decade has uncovered the biochemistry of this system. CRISPR-Cas systems consist of Cas proteins, which are involved in acquisition, targeting and cleavage of foreign DNA or RNA, and a CRISPR array, which includes direct repeats flanking short spacer sequences that guide Cas proteins to their targets. Class 2 CRISPR-Cas are streamlined versions in which a single Cas protein bound to RNA is responsible for binding to and cleavage of a targeted sequence. The programmable nature of these minimal systems has facilitated their use as a versatile technology that is revolutionizing the field of genome manipulation.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus, and include adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas-alpha protein disclosed herein, a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence.

CRISPR-Cas systems have been classified according to sequence and structural analysis of components. Multiple CRISPR/Cas systems have been described including Class 1 systems, with multisubunit effector complexes (comprising type I, type III, and type IV), and Class 2 systems, with single protein effectors (comprising type II, type V, and type VI). A CRISPR-Cas system comprises, at a minimum, a CRISPR RNA (crRNA) molecule and at least one CRISPR-associated (Cas) protein to form crRNA ribonucleoprotein (crRNP) effector complexes. CRISPR-Cas loci comprise an array of identical repeats interspersed with DNA-targeting spacers that encode the crRNA components and an operon-like unit of cas genes encoding the Cas protein components. The resulting ribonucleoprotein complex recognizes a polynucleotide in a sequence-specific manner. The crRNA serves as a guide RNA for sequence specific binding of the effector (protein or complex) to double strand DNA sequences, by forming base pairs with the complementary DNA strand while displacing the noncomplementary strand to form a so called R-loop. RNA transcripts of CRISPR loci (pre-crRNA) are cleaved specifically in the repeat sequences by CRISPR associated (Cas) endoribonucleases in type I and type III systems or by RNase III in type II systems. The number of CRISPR-associated genes at a given CRISPR locus can vary between species.

Different cas genes that encode proteins with different domains are present in different CRISPR systems. The cas operon comprises genes that encode for one or more effector endonucleases, as well as other Cas proteins. Some domains may serve more than one purpose, for example Cas9 comprises domains for endonuclease functionality as well as for target cleavage, among others. The Cas endonuclease is guided by a single CRISPR RNA (crRNA) through direct RNA-DNA base-pairing to recognize a DNA target site that is in close vicinity to a protospacer adjacent motif (PAM). Class I CRISPR-Cas systems comprise Types I, III, and IV. A characteristic feature of Class I systems is the presence of an effector endonuclease complex instead of a single protein. A Cascade complex comprises a RNA recognition motif (RRM) and a nucleic acid-binding domain that is the core fold of the diverse RAMP (Repeat-Associated Mysterious Proteins) protein superfamily.

Type I CRISPR-Cas systems comprise a complex of effector proteins, termed Cascade (CRISPR-associated complex for antiviral defense) comprising at a minimum Cas5 and Cas7. The effector complex functions together with a single CRISPR RNA (crRNA) and Cas3 to defend against invading viral DNA. Type I systems are divided into seven subtypes.

Type III CRISPR-Cas systems, comprising a plurality of cas7 genes, target either ssRNA or ssDNA, and function as either an RNase as well as a target RNA-activated DNA nuclease. Type IV systems, although comprising typical type I cas5 and cas7 domains in addition to a cas8-like domain, may lack the CRISPR array that is characteristic of most other CRISPR-Cas systems.

Class II CRISPR-Cas systems comprise Types II, V, and VI. A characteristic feature of Class II systems is the presence of a single Cas effector protein instead of an effector complex. Types II and V Cas proteins comprise an RuvC endonuclease domain that adopts the RNase H fold. Type II CRISPR/Cas systems employ a crRNA and tracrRNA (trans-activating CRISPR RNA) to guide the Cas endonuclease to its DNA target. The crRNA comprises a spacer region complementary to one strand of the double strand DNA target and a region that base pairs with the tracrRNA (trans-activating CRISPR RNA) forming a RNA duplex that directs the Cas endonuclease to cleave the DNA target, leaving a blunt end. Spacers are acquired through a not fully understood process involving Cas1 and Cas2 proteins. Type II CRISPR/Cas loci typically comprise cas1 and cas2 genes in addition to the cas9 gene. Type II CRISR-Cas loci can encode a tracrRNA, which is partially complementary to the repeats within the respective CRISPR array, and can comprise other proteins such as Csn1 and Csn2. The presence of cas9 in the vicinity of cas1 and cas2 genes is the hallmark of type II loci. Type V CRISPR/Cas systems comprise a single Cas endonuclease, including Cpf1 (Cas12) that is an active RNA-guided endonuclease that does not necessarily require the additional trans-activating CRISPR (tracr) RNA for target cleavage, unlike Cas9. Type VI CRISPR-Cas systems comprise a cas13 gene that encodes a nuclease with two HEPN (Higher Eukaryotes and Prokaryotes Nucleotide-binding) domains but no HNH or RuvC domains, and are not dependent upon tracrRNA activity. The majority of HEPN domains comprise conserved motifs that constitute a metal-independent endoRNase active site. Because of this feature, it is thought that type VI systems act on RNA targets instead of the DNA targets that are common to other CRISPR-Cas systems.

To comply with written description and enablement requirements, incorporated herein by the following references are the following patent publications: 2014/0349405 to Sontheimer; 2014/0377278 to Elinav; 2014/0068797 to Doudna; 20200190494 to Hou, et. al.; and 2020/0199555 to Zhang.

To further comply with applicable written description and enablement requirement, incorporated herein by reference is U.S. Patent Application Publication 2020/0138722, entitled “Targeted gastrointestinal tract delivery of probiotic organisms and/or therapeutic agents,” published 7 May 2020 to Kabadi et al.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” approximately,” etc. mean that each of the limitations or ranges may vary by up to about 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value between 4.5 and 5.5, the second number in the ratio can be any value between 2.7 and 3.3, and so on.

As used herein, unless otherwise specified, the term “animal” refers to any organism of the kingdom Animalia, including but not limited to a human.

As used herein, unless otherwise specified, the term “disease” refers to a disease, disorder, or condition or a symptom thereof.

As used herein, unless otherwise specified, the terms “lactobacillus” and “lactobacilli,” when used without further elaboration, refer to any organism that was, prior to the 2020 reclassification of the genus Lactobacillus into 25 distinct genera, classified as Lactobacillaceae.

As used herein, unless otherwise specified, the term “patient” refers to a mammal, including, by way of non-limiting example, a human.

As used herein, unless otherwise specified, the term “pharmaceutically acceptable” means approved or approvable by a regulatory agency of the Federal or a state government or listed in the U.S. Pharmacopoeia or other generally recognized pharmacopoeia for use in animals, and more particularly in humans.

As used herein, unless otherwise specified, the term “pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a substance provided by the present disclosure may be administered to a patient, which does not destroy the pharmacological activity thereof, and which is non-toxic when administered in doses sufficient to provide a therapeutically effective amount of the substance.

As used herein, unless otherwise specified, the term “pharmaceutical formulation” refers to a therapeutically active substance and at least one pharmaceutically acceptable vehicle, with which the substance is administered to a patient.

As used herein, unless otherwise specified, the term “prebiotic” refers to a substrate that is selectively utilized by a microorganism that confers a health benefit upon the microorganism's host.

As used herein, unless otherwise specified, the term “probiotic” refers to a live microorganism that, when administered to a host animal in adequate amounts, confers a health benefit on the host animal.

As used herein, unless otherwise specified, the term “synbiotic” refers to a combination or mixture of probiotics and prebiotics that beneficially affects a host by improving the survival and implantation of live microbial dietary supplements in the gastrointestinal tract via selective stimulation of the growth, and/or activation of the metabolism, of one or more health-promoting microbes.

As used herein, the terms “treating” and “treatment” refer to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease even though that patient does not yet experience or display symptoms of the disease.

As used herein, unless otherwise specified, the term “therapeutically effective amount” refers to the amount of a substance that, when administered to a subject for treating a disease, or at least one of the clinical symptoms of a disease, is sufficient to effect such treatment of the disease or symptom thereof. The “therapeutically effective amount” may vary depending, for example, on the substance, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

As used herein, unless otherwise specified, the term “therapeutically effective dose” refers to a dose that provides effective treatment of a disease or disorder in a patient. A therapeutically effective dose may vary from substance to substance, and from patient to patient, and may depend upon factors such as the condition of the patient and the route of delivery. A therapeutically effective dose may be determined in accordance with routine pharmacological procedures known to those skilled in the art.

The present invention provides rationally defined and assembled microbial consortia to impart health across organ systems by first modulating the function of the native gut microbiota and host tissue. More specifically, the invention provides designed oral prophylactic probiotic strain mixtures to maintain and improve host health status in local and distant body sites.

In embodiments, the present invention provides one or more consortia of live microorganisms with a range of benefits verified in humans, which may, in some embodiments, be delivered via a protective nested capsule system. Viability of the synbiotic compositions of the present invention throughout production and administration has been validated by comprehensive evaluation of Active Fluorescent Units (AFUs) in the capsule and in a simulated human intestinal environment, as described in further detail in the Examples provided below. In addition to extensive mechanistic and genetic characterization, the consortia include strains that have been clinically evaluated in strain-specific, double-blind, placebo-controlled human studies for clinical outcomes including improvement in digestion, modulation of the gut-skin axis, low-density lipoprotein modulation, gut immunological response, epithelial barrier regulation, and micronutrient synthesis.

### Microbial Strains

The development of next-generation sequencing (NGS) technologies has introduced new and much cheaper methods for prokaryotic whole genome sequencing. In combination with the decreasing price for computational resources needed for bioinformatic analyses, this has resulted in the publication of a large number of new bacterial genomes. More and more “non-bioinformatic” labs are now sequencing their own prokaryotic genomes and facing questions such as which sequencing platform to choose, how many libraries should be generated, which assembly method should be used for the libraries, etc.

One of the main questions facing each new bacterial sequencing project is whether the planned use for the genome requires it to be closed into a single, high quality sequence for each DNA molecule (genome or plasmid, defined as “complete” status). A complete genome sequence represents a finished product in which the order and accuracy of every base pair have been verified. In contrast, a draft sequence (even one of high coverage) represents a collection of contigs of various sizes, with unknown order and orientation, that contain sequencing errors and possible misassemblies. For example, if the genome in question is compared to the genomes of other organisms to infer the genetic basis for different physiological characteristics or ecological niches, such a comparison may be biased due to fragmented representation of function-related genomic regions such as operons, genomic islands or extrachromosomal elements such as plasmids. Notably, as of October 2015 the curated genome database of the National Center for Biotechnology Information (NCBI) contained 49,204 bacterial genomes, yet only 10% of them are defined as “complete genomes”. The rest are defined as “draft genomes”, which range from one contig with many N's (scaffold) to a large number of contigs whose order is unknown. The difficulty in assembling a closed genome despite the high coverage typically obtained with NGS techniques is usually caused by repetitive sequences including both highly conserved core genes (e.g., ribosomal and tRNA genes) and non-core (accessory) genes which are often harbored on mobile genetic elements such as plasmids and transposons. Such sequences often produce “break points” in the assembly, resulting in multiple contigs. Alternatively, when assembled, such multicopy genes might be collapsed into a single gene sequence, thus missing genetic microdiversity which may be functionally important. These difficulties can be largely overcome by using single-molecule, long-read sequencing technologies such as the Oxford Nanopore reads for assembly followed by polishing with Illumina reads to generate the most contiguous genomes with sufficient accuracy to enable the accurate annotation of important but difficult to sequence genomic features such as insertion sequences and secondary metabolite biosynthetic gene clusters.

Each strain in the synbiotic compositions of the present invention has been sequenced to generate closed genome assemblies using hybrid assembly of Illumina and Oxford Nanopore reads. These data allow detection of virulence factors, antibiotic resistance genes in the genomes, and plasmid-derived sequences of each strain, and allow for further customization, design, and tailoring of the compositions informed by genome-based mechanisms of action.

Embodiments of the present invention include synbiotic compositions comprising Bifidobacterium breve SD-BR3-IT. This bacterial strain may have any one or more of several beneficial effects on the health of the host. By way of first non-limiting example, Bifidobacterium breve SD-BR3-IT may improve the host's bowel movement frequency, stool consistency, and ease of expulsion, and may alleviate symptoms of intestinal discomfort such as abdominal bloating, itching, burning, or pain. By way of second non-limiting example, Bifidobacterium breve SD-BR3-IT may improve clinical parameters of atopic dermatitis as measured by SCORAD and Dermatology Life Quality (DLQ) index, reduce microbial translocation and/or immune activation, and/or improve T-helper cell (Th) 17/regulatory T cell (T_{reg}) and Th1/Th2 ratios. By way of third non-limiting example, Bifidobacterium breve SD-BR3-IT may persist in the host's gut microbiota after cessation of administration of the synbiotic, which may rectify the dysbiotic gut microbiota of atopic dermatitis patients. By way of fourth non-limiting example, Bifidobacterium breve SD-BR3-IT may inhibit the growth of multiple E. coli biotypes, including pathogenic E. coli O157:H7.

Embodiments of the present invention include synbiotic compositions comprising Lactiplantibacillus plantarum SD-LP1-IT. This bacterial strain may have any one or more of several beneficial effects on the health of the host. By way of first non-limiting example, Lactiplantibacillus plantarum SD-LP1-IT may improve the host's bowel movement frequency, stool consistency, and ease of expulsion, and may alleviate symptoms of intestinal discomfort such as abdominal bloating, itching, burning, or pain. By way of second non-limiting example, Lactiplantibacillus plantarum SD-LP1-IT may inhibit the growth of multiple E. coli biotypes, including pathogenic E. coli O157:H7.

Embodiments of the present invention include synbiotic compositions comprising Bifidobacterium longum SD-BB536-JP. This bacterial strain may have any one or more of several beneficial effects on the health of the host. By way of first non-limiting example, Bifidobacterium longum SD-BB536-JP may increase bowel movement frequency, improve fecal visual characteristics, increase fecal moisture content, decrease fecal ammonia content, decrease activity of various fecal enzymes, increase the proportion of Bifidobacteria spp. in the fecal microbiota, and/or decrease the proportion of Enterobacteriaceae spp. and/or Clostridium perfringens in the fecal microbiota. By way of second non-limiting example, Bifidobacterium longum SD-BB536-JP may decrease the incidence of respiratory illness in the host and/or cause differences in gut microbiota, including increased abundance of Faecalibacterium spp. By way of third non-limiting example, Bifidobacterium longum SD-BB536-JP may decrease the quantity of enterotoxigenic Bacteroides fragilis in the host's intestinal microbiota. By way of fourth non-limiting example, Bifidobacterium longum SD-BB536-JP may, in hosts with Japanese cedar pollinosis allergy, reduce throat and nasal symptoms such as itching, rhinorrhea, and nasal blockage, suppress allergy-induced decrease in blood levels of IFN-7 and/or increase in blood eosinophil rates, and/or decrease levels of JCP-specific IgE antibodies.

Embodiments of the present invention include synbiotic compositions comprising Limosilactobacillus reuteri SD-LRE2-IT. This bacterial strain may have any one or more of several beneficial effects on the health of the host. By way of first non-limiting example, Limosilactobacillus reuteri SD-LRE2-IT may degrade oxalate, and particularly degrade oxalate more effectively than Bifidobacteria spp. By way of second non-limiting example, Limosilactobacillus reuteri SD-LRE2-IT may produce riboflavin and vitamin B12.

Embodiments of the present invention include synbiotic compositions comprising Bifidobacterium infantis SD-M63-JP. This bacterial strain may have any one or more of several beneficial effects on the health of the host. By way of first non-limiting example, Bifidobacterium infantis SD-M63-JP may ferment human milk oligosaccharides 3′-siallyllactose, 6′-siallyllactose, 2′-fucosyllactose, and 3′-fucosyllactose. By way of second non-limiting example, Bifidobacterium infantis SD-M63-JP may, in hosts suffering from irritable bowel syndrome (IBS), lead to a lower ratio of Firmicutes spp./Bacteroidetes spp. in the gut microbiota, which correlates with improved mental wellbeing.

Embodiments of the present invention include synbiotic compositions comprising Lacticaseibacillus rhamnosus HRVD113-US. This bacterial strain may have any one or more of several beneficial effects on the health of the host. By way of non-limiting example, Lacticaseibacillus rhamnosus HRVD113-US may increase expression of markers of gut barrier integrity (including Nrf2 and epithelial tight junction proteins) and/or production of SCFAs in human intestinal epithelial cells.

Embodiments of the present invention include synbiotic compositions comprising Bifidobacterium lactis HRVD524-US (B1-04). This bacterial strain may have any one or more of several beneficial effects on the health of the host. By way of first non-limiting example, Bifidobacterium lactis HRVD524-US (B1-04) may reduce the risk of upper respiratory tract illness episodes and/or delay respiratory illness. By way of second non-limiting example, Bifidobacterium lactis HRVD524-US (B1-04) may reduce rhinovirus titers in nasal lavage, the likelihood of shedding viruses in nasal secretions, and/or chemokine (C-X-C motif) ligand 8 (CXCL8) response to rhinovirus infection in nasal lavage.

Embodiments of the present invention include synbiotic compositions comprising Bifidobacterium breve HRVD521-US. This bacterial strain may have any one or more of several beneficial effects on the health of the host. By way of non-limiting example, Bifidobacterium breve HRVD521-US may increase expression of markers of gut barrier integrity (including Nrf2 and epithelial tight junction proteins) and/or production of SCFAs in human intestinal epithelial cells.

Embodiments of the present invention include synbiotic compositions comprising Lacticaseibacillus casei HRVD300-US. This bacterial strain may have any one or more of several beneficial effects on the health of the host. By way of non-limiting example, Bifidobacterium breve HRVD521-US may increase expression of markers of gut barrier integrity (including Nrf2 and epithelial tight junction proteins) and/or production of SCFAs in human intestinal epithelial cells.

Embodiments of the present invention include synbiotic compositions comprising Bifidobacterium longum HRVD90b-US. This bacterial strain may have any one or more of several beneficial effects on the health of the host. By way of non-limiting example, Bifidobacterium longum HRVD90b-US may increase expression of markers of gut barrier integrity (including Nrf2 and epithelial tight junction proteins) and/or production of SCFAs in human intestinal epithelial cells.

Embodiments of the present invention include synbiotic compositions comprising Bifidobacterium lactis SD150-BE. This bacterial strain may have any one or more of several beneficial effects on the health of the host. By way of non-limiting example, Bifidobacterium lactis SD150-BE may increase expression of markers of gut barrier integrity (including Nrf2 and epithelial tight junction proteins) and/or production of SCFAs in human intestinal epithelial cells.

Embodiments of the present invention include synbiotic compositions comprising Lacticaseibacillus rhamnosus SD-GG-BE. This bacterial strain may have any one or more of several beneficial effects on the health of the host. By way of non-limiting example Lacticaseibacillus rhamnosus SD-GG-BE may reduce toll-like receptor mRNA levels of antigen-presenting cells (APCs), reduce CD16 expression in macrophages and CD11 expression in monocytes, and/or induce type-1 immune response polarization as observed from elevated production of IL-12 and TNF-α.

Embodiments of the present invention include synbiotic compositions comprising Limosilactobacillus reuteri RD830-FR. This bacterial strain may have any one or more of several beneficial effects on the health of the host. By way of non-limiting example, Limosilactobacillus reuteri RD830-FR may increase expression of markers of gut barrier integrity (including Nrf2 and epithelial tight junction proteins) and/or production of SCFAs in human intestinal epithelial cells.

Embodiments of the present invention include synbiotic compositions comprising Bifidobacterium adolescentis SD-BA5-IT. This bacterial strain may have any one or more of several beneficial effects on the health of the host. By way of first non-limiting example, Bifidobacterium adolescentis SD-BA5-IT may increase folic acid concentration in the host's feces and/or colonize the intestinal environment. By way of second non-limiting example, Bifidobacterium adolescentis SD-BA5-IT may produce folate.

Embodiments of the present invention include synbiotic compositions comprising Lactobacillus crispatus SD-LCR01-IT. This bacterial strain may have any one or more of several beneficial effects on the health of the host. By way of non-limiting example, Lactobacillus crispatus SD-LCR01-IT may increase expression of markers of gut barrier integrity (including Nrf2 and epithelial tight junction proteins) and/or production of SCFAs in human intestinal epithelial cells.

Embodiments of the present invention include synbiotic compositions comprising Ligilactobacillus salivarius SD-LS1-IT. This bacterial strain may have any one or more of several beneficial effects on the health of the host. By way of first non-limiting example, Ligilactobacillus salivarius SD-LS1-IT may improve clinical parameters of atopic dermatitis as measured by SCORAD and Dermatology Life Quality (DLQ) index, reduce microbial translocation and/or immune activation, and/or improve T-helper cell (Th) 17/regulatory T cell (T_{reg}) and Th1/Th2 ratios. By way of second non-limiting example, Ligilactobacillus salivarius SD-LS1-IT may decrease staphylococci count in the host's feces. By way of third non-limiting example, Ligilactobacillus salivarius SD-LS1-IT may improve clinical parameters of atopic dermatitis as measured by itch index, which may persist after cessation of synbiotic administration. By way of fourth non-limiting example, Ligilactobacillus salivarius SD-LS1-IT may decrease staphylococcal load and/or reduce production of Th2 cytokines and maintain stable production of Th1 cytokines. By way of fifth non-limiting example, Ligilactobacillus salivarius SD-LS1-IT may reduce pro-inflammatory cytokines, increase anti-inflammatory cytokines, inhibit reactive oxygen species production, restore cellular membrane integrity, and/or inhibit pathogenic E. coli and Klebsiella pneumoniae.

Embodiments of the present invention include synbiotic compositions comprising Limosilactobacillus fermentum SD-LF8-IT. This bacterial strain may have any one or more of several beneficial effects on the health of the host. By way of non-limiting example, Limosilactobacillus fermentum SD-LF8-IT may increase expression of markers of gut barrier integrity (including Nrf2 and epithelial tight junction proteins) and/or production of SCFAs in human intestinal epithelial cells.

Embodiments of the present invention include synbiotic compositions comprising Bifidobacterium longum SD-CECT7347-SP. This bacterial strain may have any one or more of several beneficial effects on the health of the host. By way of first non-limiting example, Bifidobacterium longum SD-CECT7347-SP may reduce SCORAD index and use of topical steroid treatments to treat flares of chronic dermatological disease. By way of second non-limiting example, Bifidobacterium longum SD-CECT7347-SP may reduce the altered expression of cellular proteins involved in disorganization of cell cytoskeleton, inflammation, and apoptosis in Caco-2 cells. By way of third non-limiting example, Bifidobacterium longum SD-CECT7347-SP may suppress the pro-inflammatory cytokine pattern and increase IL-10 production in peripheral blood mononuclear cells (PBMCs) in hosts with celiac disease. By way of fourth non-limiting example, Bifidobacterium longum SD-CECT7347-SP may influence phenotypic and functional maturation of monocyte-derived dendritic cells. By way of fifth non-limiting example, Bifidobacterium longum SD-CECT7347-SP may reduce cellular exhibition of toxic amino acid sequences and reduce expression of NF-κB, TNF-α, and IL-10.

Embodiments of the present invention include synbiotic compositions comprising Lacticaseibacillus casei SD-CECT9104-SP. This bacterial strain may have any one or more of several beneficial effects on the health of the host. By way of non-limiting example, Lacticaseibacillus casei SD-CECT9104-SP may reduce SCORAD index and use of topical steroid treatments to treat flares of chronic dermatological disease.

Embodiments of the present invention include synbiotic compositions comprising Bifidobacterium lactis SD-CECT8145-SP. This bacterial strain may have any one or more of several beneficial effects on the health of the host. By way of non-limiting example, Bifidobacterium lactis SD-CECT8145-SP may reduce SCORAD index and use of topical steroid treatments to treat flares of chronic dermatological disease.

Embodiments of the present invention include synbiotic compositions comprising Lactiplantibacillus plantarum SD-LPLDL-UK. This bacterial strain may have any one or more of several beneficial effects on the health of the host. By way of non-limiting example, Lactiplantibacillus plantarum SD-LPLDL-UK may reduce the host's LDL-C, total cholesterol, and/or systolic blood pressure and/or increase the host's HDL-C.

Embodiments of the present invention include synbiotic compositions comprising Bifidobacterium lactis SD-MB2409-IT. This bacterial strain may have any one or more of several beneficial effects on the health of the host. By way of non-limiting example, Bifidobacterium lactis SD-MB2409-IT may reduce total cholesterol and LDL-C and/or assimilate cholesterol and bile salt hydrolase against glycocholic and taurodeoxycholic acids.

Embodiments of the present invention include synbiotic compositions comprising Bifidobacterium lactis SD-BS5-IT. This bacterial strain may have any one or more of several beneficial effects on the health of the host. By way of non-limiting example, Bifidobacterium lactis SD-BS5-IT may promote intestinal saccharolytic metabolism and/or reduce doxorubicin-induced oxidative stress.

Embodiments of the present invention include synbiotic compositions comprising Lacticaseibacillus rhamnosus SD-LR6-IT. This bacterial strain may have any one or more of several beneficial effects on the health of the host. By way of non-limiting example, Lacticaseibacillus rhamnosus SD-LR6-IT may inhibit the growth of multiple E. coli biotypes, including pathogenic E. coli O157:H7.

Aspects of the present invention allow for rational and systematic screening and selection of bacterial strains of interest for use in synbiotic compositions. Particularly, as described throughout this disclosure, bacterial strains of interest can be, and in preferred embodiments are, screened for a variety of functional attributes, including but by no means limited to upregulation of Nrf2 transcription factor, increased short-chain fatty acid (SCFA) production in the gut, and/or improved epithelial barrier function in the gut. As a result of this screening, individual strains or combinations of strains, each possessing one or more of these desired functional attributes and collectively possessing most or all of the desired attributes, can be included in the synbiotic composition to provide a synergistic effect on the health of the host. By way of non-limiting example, upregulation of Nrf2, increased SCFA production, and improved epithelial barrier function may, in some embodiments, mutually reinforce each other in the gut environment of the host and may therefore result in a greater improvement in overall host health than any one or two of these functional outcomes; thus, strains may be screened for these attributes and rationally selected for inclusion in the synbiotic compositions of the invention as a result of this screening.

### Prebiotics

The majority of prebiotics used in conventional prebiotic dietary supplements are certain microbe-fermentable dietary fibers, i.e. polymeric and oligomeric carbohydrates that are utilized by gut microbes to support growth and promote microbial production of beneficial compounds through the multistep fermentation process. Common examples of such fibers include fructo-oligosaccharides (FOSs), galacto-oligosaccharides (GOSs), and inulin. However, to deliver a substantial health benefit, these bacteria-fermentable fiber substrates are typically required in amounts greater than can be provided in a single capsule or other dosage form, and so are instead best obtained from food sources, e.g. nuts, fruits, and vegetables. Thus, it is preferable to provide an alternative type of prebiotic in prebiotic dietary supplements.

Embodiments of the present invention include synbiotic compositions comprising polyphenols that may be biotransformed by the microbiota into one or more metabolites. The polyphenols are a large and heterogeneous group of compounds characterized by hydroxylated phenyl moieties, and are found mostly in plants, including fruits, vegetables, and cereals, or in plant-derived beverages, such as tea, coffee, and wine. Polyphenols have recently become the focus of much research due to their potential health benefits, particularly in relation to the prevention of cancer and cardiovascular disease. These plant-derived molecules are not processed by fermentation, but instead are biotransformed by gut microbes into specific metabolites that may be useful for the human body. Scientific evidence suggests that polyphenols can modulate the composition of gut microbiota in human hosts, improving a variety of biochemical markers and risk factors for chronic diseases.

More particularly, embodiments of the present invention include synbiotic compositions comprising punicalagins, which may in some embodiments be derived or extracted from pomegranates. Punicalagins are potent polyphenols found in the fruit and skin of pomegranates that provide the fruit's deep burgundy color. In the human body, punicalagins act as powerful antioxidants and can be metabolized by certain gut bacteria into a class of dibenzopyran-6-ones known as urolithins, including but not necessarily limited to urolithin-A. Urolithin-A may have any one or more of several beneficial effects on the health of the host. By way of non-limiting example, urolithin-A drives the process of mitophagy, i.e. the recycling of defective mitochondria, and therefore can have a profound positive effect on the metabolic health of the host.

While the embodiments of the invention disclosed herein are generally directed toward synbiotic compositions comprising punicalagins as the primary constituent of the prebiotic component, it is to be expressly understood that any compound that can be converted, by a microbial strain present in the healthy human gut microbiota, into a bioactive metabolite may be provided in addition to, or instead of, punicalagins. By way of first non-limiting example, the prebiotic component may comprise one or more glucosinolates, which may be metabolized by the gut microbiota into isothiocyanates, which in turn have been shown to have anti-cancer properties and other beneficial roles in human health. By way of second non-limiting example, the prebiotic component may comprise one or more catechins, which may be metabolized by microbes in the colon into γ-valerolactones and hippuric acids, which may in turn be biotransformed in the human liver into metabolites useful for human health. By way of third non-limiting example, the prebiotic component may comprise one or more polyphenols, many of which are known to be metabolized by the gut microbiota into compounds that are beneficial for human health. These and other embodiments are within the scope of the present invention. In some embodiments, the compound may be a compound that is otherwise, from the host's perspective, biologically “inert” (i.e. can be metabolized only by one or more strains in the gut microbiota and not by the host), whereas in other embodiments the compound can be metabolizable by both the host and the gut microbiota.

### Delivery Capsule

Many conventional dietary probiotic compositions in the form of swallowable capsules suffer from poor targeting of the composition to the human host. Particularly, it has proven difficult in these conventional capsule compositions to provide a capsule that can ensure the survival of a large proportion of the live microorganisms through the harsh conditions of the upper digestive tract and then release the live microorganisms to the lower digestive tract, where they are most beneficial. In many cases, conventional capsules are degraded in the upper digestive tract to such an extent that a large proportion of the live microorganisms are destroyed before reaching the lower digestive tract.

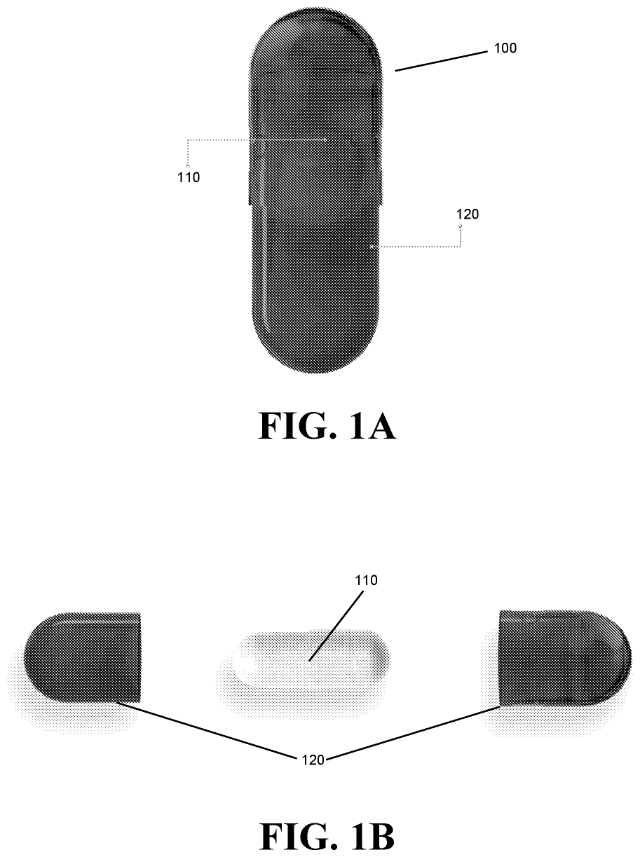

Referring now to FIGS. 1A and 1i, embodiments of the present invention include an improved capsule for delivery of probiotic compositions. As illustrated in assembled view in FIGS. 1A and 1n exploded view in FIG. 1B, a delivery capsule 100 includes an inner capsule 110 enclosed within, embedded within, and/or surrounded by an outer capsule 120. While the compositions and structures of the inner 110 and outer 120 capsules may vary, in many embodiments the inner capsule 110 will contain or enclose most or all of a probiotic component of a synbiotic composition, while the outer capsule 120 contains or encloses most or all of a prebiotic component of the synbiotic composition. This delivery capsule 100 allows for improved stability and viability through the gastrointestinal tract, resulting in 100% or nearly 100% survival of the probiotic organisms through the stomach, jejunum, duodenum, and ileum until arriving in the colon, as described in greater detail in the Examples below. Distinctly, the delivery capsule 100 of the present invention allows for precision release in the upper small intestine, thereby increasing engagement between the organisms and host cells, in contrast to traditional delayed-release or enteric coating-based acid-resistant delivery systems. Without wishing to be bound by any particular theory, the outer capsule 120 may degrade or be digested, thereby resulting in release of the prebiotic component of the synbiotic composition, before the inner capsule 110 is degraded or digested to result in release of the probiotic component of the synbiotic composition. An important advantage of the delivery capsule 100 of the present invention is that it allows for improved release characteristics of either or both of the prebiotic component and the probiotic component, and in particular improved survival of the probiotic component through the upper digestive system until release in the colon, without the need for separate liquid media or solvents.

One advantage of the “capsule-in-capsule” construction of the capsule 100 illustrated in FIGS. 1A and 1B is that both the inner 110 and outer 120 capsule can be constructed of conventional excipients, tableting ingredients, controlled-delivery component materials, etc. The inner 110 and outer capsules 120 may be made of the same materials or different materials. By way of non-limiting example, in many embodiments, both the inner 110 and outer 120 capsule may be constructed of hypromellose; the inner capsule 110 may be constructed of the same grade, or a different grade, of hypromellose as the outer capsule 120.

### Pharmaceutical Formulations

Pharmaceutical formulations provided by the present disclosure may comprise a therapeutically effective amount of a synbiotic composition together with a suitable amount of one or more pharmaceutically acceptable vehicles so as to provide a formulation for proper administration to a patient. Suitable pharmaceutical vehicles are described in the art.

In certain embodiments, the synbiotic composition may be incorporated into pharmaceutical formulations to be administered orally. Oral administration of such pharmaceutical formulations may result in release and/or uptake of the synbiotic composition throughout the intestine. Such oral formulations may be prepared in a manner known in the pharmaceutical art and comprise the synbiotic composition and at least one pharmaceutically acceptable vehicle. Oral pharmaceutical formulations may include a therapeutically effective amount of the synbiotic composition and a suitable amount of a pharmaceutically acceptable vehicle, so as to provide an appropriate form for administration to a patient.

The synbiotic composition may be incorporated into pharmaceutical formulations to be administered by any other appropriate route of systemic administration including intramuscular, intravenous and oral.

Pharmaceutical formulations comprising a synbiotic composition may be manufactured by means of conventional mixing, dissolving, granulating, dragee-making, levigating, emulsifying, encapsulating, entrapping, or lyophilizing processes. Pharmaceutical formulations may be formulated in a conventional manner using one or more physiologically acceptable carriers, diluents, excipients, or auxiliaries, which facilitate processing of the synbiotic composition and one or more pharmaceutically acceptable vehicles into formulations that can be used pharmaceutically. Proper formulation is dependent upon the route of administration chosen. Pharmaceutical formulations provided by the present disclosure may take the form of sustained-release formulations suitable for administration to a patient.

Pharmaceutical formulations provided by the present disclosure may be formulated in a unit dosage form. A unit dosage form refers to a physically discrete unit suitable as a unitary dose for patients undergoing treatment, with each unit containing a predetermined quantity of the synbiotic composition calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a patient at a single point in time or during a time interval.

In certain embodiments, an oral dosage form provided by the present disclosure may be a controlled release dosage form. Controlled delivery technologies can improve the absorption of an active ingredient in a particular region or regions of the gastrointestinal tract. Controlled active ingredient delivery systems may be designed to deliver an active ingredient in such a way that the level of the active ingredient is maintained within a therapeutically effective window and effective and safe blood levels are maintained for a period as long as the system continues to deliver the active ingredient with a particular release profile in the gastrointestinal tract. Controlled active ingredient delivery may produce substantially constant blood levels of an active ingredient over a period of time as compared to fluctuations observed with immediate release dosage forms. For some applications, maintaining a constant blood and tissue concentration throughout the course of therapy is the most desirable mode of treatment. Immediate release of an active ingredient may cause blood levels to peak above the level required to elicit a desired response, which may waste the active ingredient and may cause or exacerbate toxic side effects. Controlled active ingredient delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

An appropriate oral dosage form for a particular pharmaceutical formulation provided by the present disclosure may depend, at least in part, on the gastrointestinal absorption properties of the active ingredient and/or the stability of the active ingredient in the gastrointestinal tract, the pharmacokinetics of the active ingredient and the intended therapeutic profile. An appropriate controlled release oral dosage form may be selected for a particular ingredient or combination of ingredients. For example, gastric retention oral dosage forms may be appropriate for active ingredients absorbed primarily from the upper gastrointestinal tract, and sustained release oral dosage forms may be appropriate for active ingredients absorbed primarily from the lower gastrointestinal tract. Certain active ingredients are absorbed primarily from the small intestine. In general, active ingredients traverse the length of the small intestine in about 3 to 5 hours. For active ingredients that are not easily absorbed by the small intestine or that do not dissolve readily, the window for active agent absorption in the small intestine may be too short to provide a desired therapeutic effect.

In certain embodiments, pharmaceutical formulations provided by the present disclosure may be practiced with dosage forms adapted to provide sustained release of synbiotic compositions upon oral administration. Sustained release oral dosage forms may be used to release active ingredients over a prolonged time period and are useful when it is desired that an active ingredient be delivered to the lower gastrointestinal tract, including the colon. Sustained release oral dosage forms include any oral dosage form that maintains therapeutic concentrations of an active ingredient in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

In certain embodiments, pharmaceutical compositions provided by the present disclosure may include any enteric-coated sustained release oral dosage form for administering the synbiotic composition. In one embodiment, the enteric-coated oral dosage form is administered to a patient at a dosing frequency of three times per day. In another embodiment, the enteric-coated oral dosage form is administered to a patient at a dosing frequency of twice per day. In still another embodiment, the enteric-coated oral dosage form is administered to a patient at a dosing frequency of once per day.

In certain embodiments, pharmaceutical formulations provided by the present disclosure may include any non enteric-coated sustained release oral dosage form for administering the synbiotic composition. In one embodiment, the non enteric-coated oral dosage form is administered to a patient at a dosing frequency of three times per day. In another embodiment, the non enteric-coated oral dosage form is administered to a patient at a dosing frequency of twice per day. In still another embodiment, the non enteric-coated oral dosage form is administered to a patient at a dosing frequency of once per day.

In certain embodiments, pharmaceutical formulations provided by the present disclosure may include any capsule oral dosage form for administering the synbiotic composition. In one embodiment, the capsule oral dosage form is administered to a patient at a dosing frequency of three times per day. In another embodiment, the capsule oral dosage form is administered to a patient at a dosing frequency of twice per day. In still another embodiment, the capsule oral dosage form is administered to a patient at a dosing frequency of once per day.

In certain embodiments, pharmaceutical formulations provided by the present disclosure may include any suitable dosage forms that achieve the above described in vitro release profiles. Such dosage forms may be any systemic dosage forms, including sustained release enteric-coated oral dosage form and sustained release enteric-coated or non-enteric-coated oral dosage form. Examples of suitable dosage forms are described herein. Those skilled in the formulation art can develop any number of acceptable dosage forms given the dosage forms described in the examples as a starting point.

An appropriate dose of the synbiotic composition may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

### Uses

The methods and formulations disclosed herein can be used to treat patients suffering from diseases, disorders, conditions, and symptoms for which synbiotic compositions are known to provide or are later found to provide therapeutic benefit. Formulations disclosed herein can be used to treat a disease chosen from adrenal leukodystrophy, AGE-induced genome damage, Alexanders Disease, alopecia areata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome.

Methods of treating a disease in a patient provided by the present disclosure comprise administering to a patient in need of such treatment a therapeutically effective amount of a synbiotic composition of the invention. These methods and pharmaceutical formulations provide therapeutic or prophylactic amounts of the prebiotic compounds and/or probiotic strains following administration to a patient. The synbiotic composition may be administered in an amount and using a dosing schedule as appropriate for treatment of a particular disease.

Daily doses of compounds of the prebiotic component of the synbiotic composition may range from about 0.01 mg/kg to about 50 mg/kg, from about 0.1 mg/kg to about 50 mg/kg, from about 1 mg/kg to about 50 mg/kg, and in certain embodiments, from about 5 mg/kg to about 25 mg/kg. In certain embodiments, compounds of the prebiotic component may be administered at a dose over time from about 1 mg to about 5 g per day, from about 10 mg to about 4 g per day, in certain embodiments from about 20 mg to about 2 g per day, in certain embodiments from about 100 mg to about 1 g per day, in certain embodiments from about 150 mg to about 650 mg per day, in certain embodiments from about 250 mg to about 550 mg per day, in certain embodiments from about 350 mg to about 450 mg per day, and in certain embodiments about 400 mg per day.

In certain embodiments, microbial strains of the probiotic component may be administered at a dose over time from about 125 million AFU to about 625 billion AFU per day, in certain embodiments from about 1.25 billion AFU to about 500 billion AFU per day, in certain embodiments from about 2.5 billion AFU to about 250 billion AFU per day, in certain embodiments from about 12.5 billion AFU to about 125 billion AFU per day, in certain embodiments from about 25 billion AFU to about 100 billion AFU per day, in certain embodiments from about 37.5 billion AFU to about 75 billion AFU per day, and in certain embodiments from about 50 billion AFU to about 62.5 billion AFU per day.

In some embodiments, the probiotic component may comprise at least about 2.5 billion AFU per capsule, and/or at least about 5 billion AFU per dose, of Bifidobacterium breve SD-BR3-IT. In some embodiments, the probiotic component may comprise at least about 2.5 billion AFU per capsule, and/or at least about 5 billion AFU per dose, of Lactiplantibacillus plantarum SD-LP1-IT. In some embodiments, the probiotic component may comprise at least about 6 billion AFU per capsule, and/or at least about 12 billion AFU per dose, of Lacticaseibacillus rhamnosus SD-LR6-IT. In some embodiments, the probiotic component may comprise at least about 2.5 billion AFU per capsule, and/or at least about 5 billion AFU per dose, of Bifidobacterium adolescentis SD-BAS-IT. In some embodiments, the probiotic component may comprise at least about 1 billion AFU per capsule, and/or at least about 2 billion AFU per dose, of Ligilactobacillus salivarius SD-LS1-IT. In some embodiments, the probiotic component may comprise at least about 2.4 billion AFU per capsule, and/or at least about 4.8 billion AFU per dose, of Limosilactobacillus reuteri SD-LRE2-IT. In some embodiments, the probiotic component may comprise at least about 425 million AFU per capsule, and/or at least about 850 million AFU per dose, of Bifidobacterium lactis SD-BSS-IT. In some embodiments, the probiotic component may comprise at least about 500 million AFU per capsule, and/or at least about 1 billion AFU per dose, of Bifidobacterium lactis SD-MB2409-IT. In some embodiments, the probiotic component may comprise at least about 425 million AFU per capsule, and/or at least about 850 million AFU per dose, of Lactobacillus crispatus SD-LCR01-IT. In some embodiments, the probiotic component may comprise at least about 85 million AFU per capsule, and/or at least about 170 million AFU per dose, of Limosilactobacillus fermentum SD-LF8-IT. In some embodiments, the probiotic component may comprise at least about 4.25 billion AFU per capsule, and/or at least about 8.5 billion AFU per dose, of Lacticaseibacillus rhamnosus HRVD113-US. In some embodiments, the probiotic component may comprise at least about 1 billion AFU per capsule, and/or at least about 2 billion AFU per dose, of Bifidobacterium lactis HRVD524-US (B1-04). In some embodiments, the probiotic component may comprise at least about 300 million AFU per capsule, and/or at least about 600 million AFU per dose, of Lacticaseibacillus casei HRVD300-US. In some embodiments, the probiotic component may comprise at least about 300 million AFU per capsule, and/or at least about 600 million AFU per dose, of Bifidobacterium breve HRVD521-US. In some embodiments, the probiotic component may comprise at least about 120 million AFU per capsule, and/or at least about 240 million AFU per dose, of Bifidobacterium longum HRVD90b-US. In some embodiments, the probiotic component may comprise at least about 3 billion AFU per capsule, and/or at least about 6 billion AFU per dose, ofBifidobacterium longum SD-BB536-JP. In some embodiments, the probiotic component may comprise at least about 250 million AFU per capsule, and/or at least about 500 million AFU per dose, of Bifidobacterium infantis SD-M63-JP. In some embodiments, the probiotic component may comprise at least about 60 million AFU per capsule, and/or at least about 120 million AFU per dose, of Bifidobacterium lactis SD150-BE. In some embodiments, the probiotic component may comprise at least about 20 million AFU per capsule, and/or at least about 40 million AFU per dose, of Lacticaseibacillus rhamnosus SD-GG-BE. In some embodiments, the probiotic component may comprise at least about 20 million AFU per capsule, and/or at least about 40 million AFU per dose, of Limosilactobacillus reuteri RD830-FR. In some embodiments, the probiotic component may comprise at least about 210 million AFU per capsule, and/or at least about 420 million AFU per dose, of Bifidobacterium lactis SD-CECT8145-SP. In some embodiments, the probiotic component may comprise at least about 210 million AFU per capsule, and/or at least about 420 million AFU per dose, of Bifidobacterium longum SD-CECT7347-SP. In some embodiments, the probiotic component may comprise at least about 180 million AFU per capsule, and/or at least about 360 million AFU per dose, of Lacticaseibacillus casei SD-CECT9104-SP. In some embodiments, the probiotic component may comprise at least about 2.465 billion AFU per capsule, and/or at least about 4.93 billion AFU per dose, of Lactiplantibacillus plantarum SD-LPLDL-UK. In these embodiments, the absolute AFU counts of any one or more strains present in the probiotic component may vary, so long as the AFU ratio between any two or more selected microbial strains remains about the same as set forth above (for example, so long as the AFU ratio between Bifidobacterium breve SD-BR3-IT and Lacticaseibacillus rhamnosus SD-LR6-IT remains about 2.5 billion. 6 billion, or about 5 billion: 12 billion.

An appropriate dose of synbiotic composition may be determined based on several factors, including, for example, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

The synbiotic composition may be assayed in vitro and in vivo for the desired therapeutic or prophylactic activity prior to use in humans. In vivo assays, for example using appropriate animal models, may also be used to determine whether administration of synbiotic composition is therapeutically effective.

In certain embodiments, a therapeutically effective dose of the synbiotic composition may provide therapeutic benefit without causing substantial toxicity including adverse side effects. Toxicity of the synbiotic composition and/or metabolites thereof may be determined using standard pharmaceutical procedures and may be ascertained by those skilled in the art. The dose ratio between toxic and therapeutic effect is the therapeutic index. A dose of the synbiotic composition may be within a range capable of establishing and maintaining a therapeutically effective circulating plasma and/or blood concentration of, e.g., a prebiotic ingredient that exhibits little or no toxicity.

Synbiotic composition administration may be used to treat a disease chosen from adrenal leukodystrophy, AGE-induced genome damage, Alexanders Disease, alopecia areata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome. The underlying etiology of any of the foregoing diseases being treated may have a multiplicity of origins. Further, in certain embodiments, a therapeutically effective amount of synbiotic composition may be administered to a patient, such as a human, as a preventative measure against the foregoing diseases and disorders. Thus, a therapeutically effective amount of synbiotic composition may be administered as a preventative measure to a patient having a predisposition for and/or history of adrenal leukodystrophy, AGE-induced genome damage, Alexanders Disease, alopecia areata, Alper's Disease, Alzheimer's disease, amyotrophic lateral sclerosis, angina pectoris, arthritis, asthma, balo concentric sclerosis, Behcet's disease, bollus pemphigoid, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-xB mediated diseases, optic neuritis, pareneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinopathia pigmentosa, sarcoidosis, Schilders Disease, subacute necrotizing myelopathy, susac syndrome, transplantation rejection, transverse myelitis, a tumor, ulcerative colitis or Zellweger's syndrome.

In some embodiments, methods and compositions of the invention may be effective for metagenomic and metabolomic reconstitution of gut microbiota after broad spectrum antibiotic therapy. Additional disclosure regarding such methods and compositions may be found in Clinicaltrials.gov Study NCT04171466, “Metagenomic and Metabolomic Reconstitution of Gut Microbiota After Broad Spectrum Antibiotic Therapy,” the entirety of which is incorporated herein by reference.

In some embodiments, methods and compositions of the invention may be effective to restore epithelial barrier integrity and gut microbiota function after broad spectrum antibiotic therapy. Additional disclosure regarding such methods and compositions may be found in Clinicaltrials.gov Study NCT04171466, “Metagenomic and Metabolomic Reconstitution of Gut Microbiota After Broad Spectrum Antibiotic Therapy,” the entirety of which is incorporated herein by reference.

In some embodiments, methods and compositions of the invention may be effective to improve metagenomic stability and metabolic output of the gut microbiota in subjects with irritable bowel syndrome.

In some embodiments, methods and compositions of the invention may be effective to improve, remediate, or restore function to the gut and airway microbiomes of adults with mild to moderate COVID-19.

In some embodiments, methods and compositions of the invention may be effective to retain gut barrier integrity and gut microbiota composition after rifaximin treatment in subjects with irritable bowel syndrome.

In some embodiments, methods and compositions of the invention may be effective to ameliorate, prevent, or treat constipation.

The invention is further described by reference to the following non-limiting Examples. The Examples illustrate various aspects of the disclosure. It will be apparent to those skilled in the art that many modifications, to both materials and methods, may be practiced without departing from the scope of the disclosure.

### Example 1

### Whole Genome Shotgun Sequencing

Metagenomic DNA was extracted from the probiotic component of synbiotic compositions of the present invention using the Mag Attract Power Soil kit (Qiagen, Cat #27000-4-EP). Sequence libraries were prepared using the Nextera DNA Flex protocol (Illumina). Sequencing was performed on the HiSeq3000 (2×150 bp), generating 22.27 Gb for both R1 and R2 reads prior to quality filtering. Sequences were filtered using Trimmomatic, under default settings and with a minimum sequence length of 75 bp. Taxonomic composition was determined using MetaPh1An2.

A read-level analysis was performed in which the pair-end sequence reads from the probiotic mixture were mapped to the most recent versions of two publicly-available AMR databases: ResFinder (Apr. 26, 2019 release) and the NCBI Antimicrobial Resistance database (Apr. 29, 2019 release) using kma. The kma algorithm is a rapid and precise kmer-based aligner designed to map raw (i.e., non-assembled) sequence reads and identify hits within redundant databases. The kma aligner produces outputs that are much like a traditional BLAST report, except that they are database-focused rather than query-focused and present evidence related to identity, coverage, and the depth of coverage represented by all hits identified in the pool of unassembled reads.

A total of 22.27 Gb of raw sequence data were produced. After quality filtering, 16.04 Gb of data were utilized for taxonomic and AMR profiling. Taxonomic profiling identified 12 bacterial species with Bifidobacterium longum, Bifidobacterium animalis, Lacticaseibacillus rhamnosus, Lactiplantibacillus plantarum, Ligilactobacillus salivarius, and Bifidobacterium breve accounting for the majority of the sequence reads identified in the community.

### Example 2

### Screening Against Nrf2 Transcription Factor

The invertebrate Caenorhabditis elegans was the first multicellular organism to have its whole genome sequenced, and a combination of a short 2 to 3 week lifespan, a transparent cell wall, and genetic tractability have made it an extremely versatile model system used to study energy metabolism, immunity, and aging. Whereas the C. elegans worm demonstrates a diverse microbiota in the wild, it is typically grown with one species in the lab; this enables researchers to easily create and maintain a defined intestinal microbiota. The intestines are one of the organisms' major organs and constitute roughly a third of their somatic mass. The transparent cell wall and aerobic lumen also enable the simple visualization of fluorescent proteins and markers. The worm possesses an innate immune system that is used to regulate the intestinal bacterial load as the worm ages. The emerging need for both convenient and robust tools to investigate host-microbiota interactions has resulted in a growing interest in the use of C. elegans as a live animal model for both host-microbiota interactions and synthetic biology. Examples include high-throughput screens to elucidate the complexity of underlying host-microbe-drug interactions, understanding how bacterial-produced metabolites affect the worm gene expression and its lifespan, and the role of stochasticity in the colonization of the gut by microbiota.

Cellular damage caused by reactive oxygen species is believed to be a major contributor to age-associated diseases. Humans have developed a robust response pathway to respond to environmental stressors and produce a range of detoxification enzymes in response to xenobiotics. The gastrointestinal tract, alongside the skin, is a high contact surface with external stressors, and consequently regulates the host's immunological response. Recently, the use of microbes to engage with this system and affect localized and systemic transcriptional pathways has been explored. Identifying the signaling mechanisms and genetic factors involved in cellular surveillance-activated detoxification and immune response can identify novel probiotics for its modulation and prevent or treat adverse outcomes.

Microbial strains present in probiotic components of the synbiotic compositions of the present invention were first evaluated as single hits and in consortia in a novel C. elegans based model to identify upregulation of host detoxification response, via a pathway known as Skn-1, the functional ortholog of mammalian Nrf2 transcription factor. Nrf2 is a biologically relevant pathway because it induces the expression of antioxidants as well as cytoprotective genes, which collectively provoke an anti-inflammatory expression profile and modulate inflammasome response.

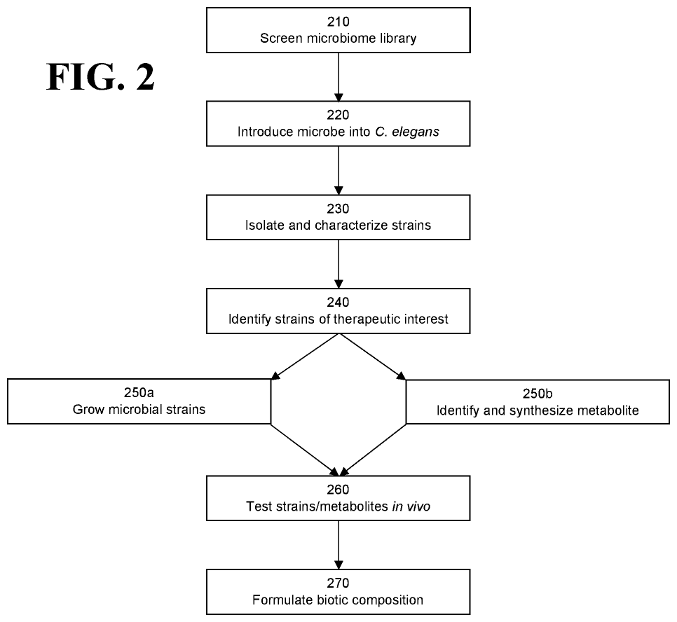

Referring now to FIG. 2, a method 200 for identifying and validating inhibitor microorganism consortia or components thereof for attenuation of detoxification and immune response and/or treatment of related symptoms is illustrated. In a first step 210 of the method 200, a microbiome library, comprising information pertaining to approximately 1,400 unique microbes and over 10 million gene activities, is screened to identify a microbe of interest. In a second step 220 of the method 200, the microbe of interest is introduced into any one of over 50 ready C. elegans models, e.g. for diabetes, apoptosis, cancer, etc. In a third step 230 of the method 200, individual microbial strains are isolated from the C. elegans model and characterized, and in a fourth step 240 of the method 200, strains of therapeutic interest are identified by genetic screening. These strains of therapeutic interest are then subjected to one or both of a growing step 250a, in which the fully characterized microbial strain is grown by any suitable method, and/or a metabolite identification step 250b, in which a microbial metabolite, i.e. a potential active therapeutic ingredient, is identified and synthesized. The microbial strain in step 250a and/or the microbial metabolite synthesized in step 250b may then be tested in animal models and/or patient tissues in step 260 to assess the therapeutic effect of the microbial strain (as a probiotic), the microbial metabolite (as a prebiotic or postbiotic), or both (as a synbiotic composition). The results of animal and/or patient tissue testing step 260 may then be used to rationally select strains and/or compounds for use in precision prebiotic, probiotic, postbiotic, and/or synbiotic compositions in formulation step 270.

Microbial strains present in probiotic components of the synbiotic compositions of the present invention were screened in isolation against C. elegans for cytoprotective responses. To prevent damage caused by environmental stresses, organisms have developed defense mechanisms that ultimately result in protective responses. Oxidative stress is generated in cells due to increased reactive oxygen species (ROS) production and is associated with increased protein and lipid damage and reduced cellular function. In addition to gastrointestinal distress, elevated levels of ROS are linked to aging, neurodegeneration, and diabetes. Nrf2 is a redox sensitive transcription factor that mediates adaptive responses to cellular stress and protection against endogenous and environmental stressors.

Nrf2 regulates the expression of a number of detoxifying enzymes including glutathione S transferases (GST) that help the system to detoxify ROS and ROS-induced cellular changes. SKN-1 is the C. elegans functional ortholog of the mammalian Nrf transcription factors and activation of SKN-1 induces the expression of GST-4, which is a homolog of glutathione S transferases (GST). SKN-1 activation was assayed by assessing the expression of gst-4 gene, which is hooked to GFP protein (gst-4::GFP). Whenever SKN-1 is activated, gst-4::GFP expression is increased. In normal or untreated animals, weakgst-4::GFP expression is observed in the body wall muscles and skin tissues.

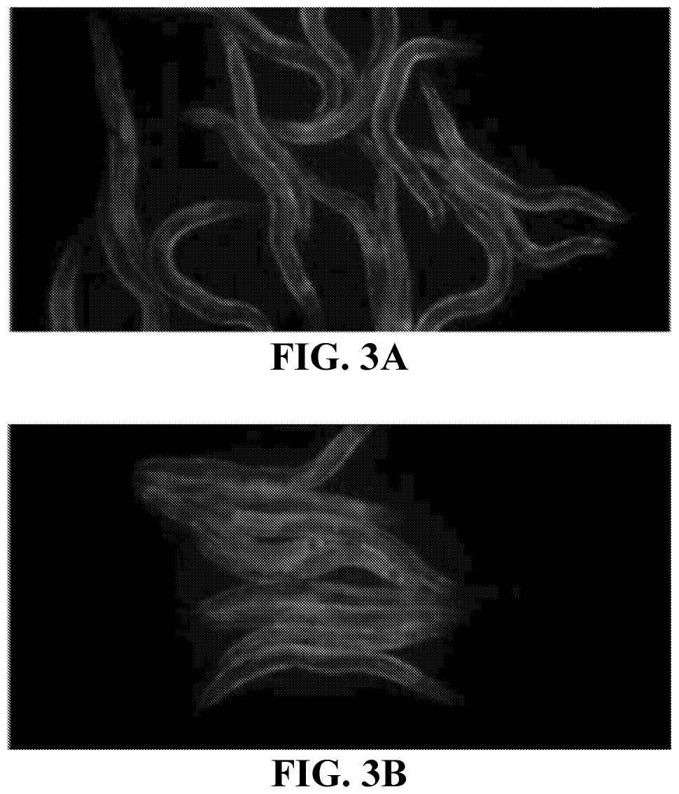

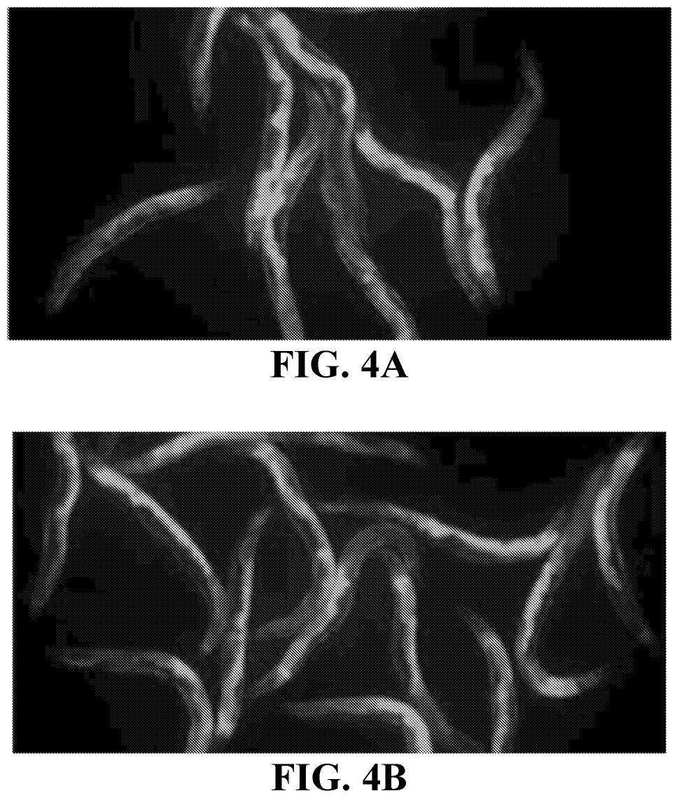

Referring now to FIGS. 3A through 4B, results from the combined treatment of microbial consortia of the probiotic components of synbiotic compositions of the present invention are illustrated. As illustrated in FIGS. 3A and 3B (representing two separate untreated C. elegans replicates), weak gst-4::GFP was observed. As illustrated in FIGS. 4A and 4B (representing two separate C. elegans replicates treated with the microbial consortia of the present invention), gst-4::GFP is clearly greatly strengthened and increased. Thus, without wishing to be bound by any particular theory, it is believed that administration of the microbial consortia of the probiotic components of synbiotic compositions of the present invention may improve the mediation of a host's adaptive responses to cellular stress and/or protect the host against endogenous and environmental stressors by upregulating Nrf2, and may thereby be effective to ameliorate, prevent, or treat any one or more of gastrointestinal distress, aging, neurodegeneration, and diabetes.

### Example 3

### Effect of Nrf2 Upregulation on Tight Junction Proteins

Nrf2 is important for the expression of genes required for maintaining proper epithelial barrier function. Upregulation of the Nrf2 pathway in the intestinal epithelial cells has been shown to reduce ROS levels, enhance enterocyte survival, and increase the integrity of tight junctions. Tight junction proteins are represented by claudins, occludin, junctional adhesion molecules, and scaffold protein zonula occludens. Among these tight junction proteins, claudins are the major components of tight junctions and are responsible for the barrier and the polarity of epithelial cells. Gastrointestinal diseases including reflux esophagitis, inflammatory bowel disease, functional gastrointestinal disorders, and cancers may be regulated by these molecules, and disruption of their functions leads to chronic inflammatory conditions and chronic or progressive disease.

To further probe the mechanisms by which the synbiotic compositions of the present invention affect tight junction proteins, experiments were performed with the colon-carcinoma cell line HT-29/B6 grown at 37° C. in a 5% CO2 atmosphere. HT-29/B6 cells originally subcloned from HT-29 cells were cultivated in RPMI 1640 medium (Biochrom) containing 2% stabilized L-glutamine and supplemented with 10% (v/v) FCS. Six isolates of these cells were obtained, of which five were treated with a synbiotic composition according to the present invention and one was left untreated as a control.

The antibody against claudin-1 was obtained from Zymed Laboratories Inc. For western blot analyses cell lysates were separated by SDS-PAGE and transferred to polyvinylidene difluoride (PVDF) membranes (PolyScreen, Perkin Elmer Life Sciences) by semidry transfer. Membranes were blocked in TST buffer (10 mM Tris-HCl pH 7.5, 150 mM NaCl, 0.1% (v/v) Tween 20) for 1 hour at room temperature (RT). Incubation with the first antibody at a concentration of 1 μg/ml in TST was performed at RT for 1 hour. After three washes with TST, membranes were incubated for 30 minutes with horseradish peroxidase-conjugated second antibody diluted 1:10,000 in TST and subsequently washed. Chemiluminescence detection was performed by exposure of Biomax MR films (Kodak, Rochester, NY) to LumiLight western blotting substrate-treated membranes. The molecular masses of the protein fragments were determined using the BenchMark™ Prestained Protein Ladder (Invitrogen Life Technologies, Karlsruhe, Germany).

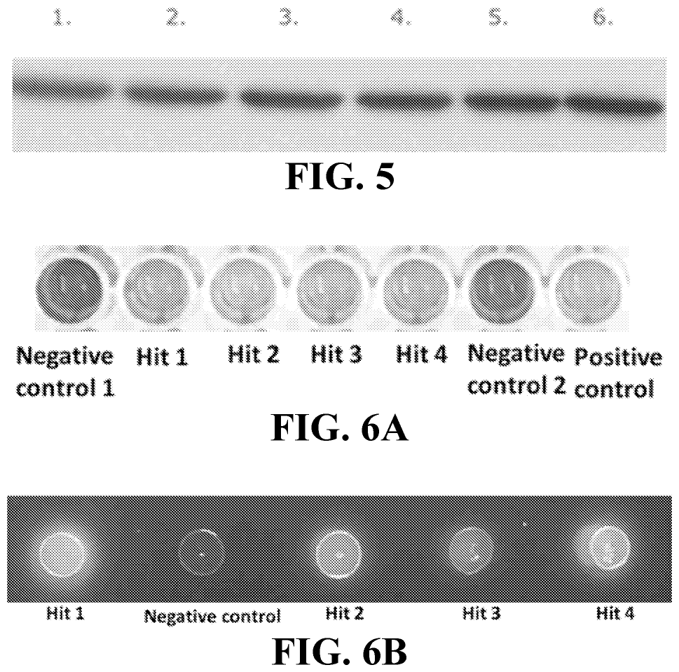

Referring now to FIG. 5, the western blot results are illustrated; in FIG. 5, the isolate labeled “1” is the control (untreated) isolate, while the isolates labeled “2” through “6” are isolates treated with the synbiotic composition. As FIG. 5 illustrates, compared to untreated cells, increased expression of claudin-1 is observed after treatment of intestinal epithelial cells with microbial strains of the probiotic component of synbiotic compositions of the present invention that upregulate Nrf2. This conclusion is further reinforced by densitometric values of the increase in expression of tight junction protein, given in Table 1 below.

TABLE 1

| | % increase in tight junction protein |
|---|---|
| Isolate # | relative to control |
| 1 (control) | 0 |
| 2 | 102 |
| 3 | 89 |
| 4 | 59 |
| 5 | 26 |
| 6 | 35 |

Thus, without wishing to be bound by any particular theory, it is believed that administration of the synbiotic compositions of the present invention may improve epithelial barrier function by upregulating Nrf2, and may thereby be effective to ameliorate, prevent, or treat any one or more gastrointestinal diseases.

### Example 4

### Organic Acid Production

Organic acid production is an important feature by which microorganisms modulate the metabolic output of the gut microbiota and engage with host cells. Organic acids, in particular short-chain fatty acids (SCFAs), are bacterial fermentation products that are chemically composed of a carboxylic acid moiety and a small hydrocarbon chain. Among the SCFAs, acetic, propionic, and butyric acids are the most studied, presenting two, three, and four carbon atoms in their chemical structures, respectively. These metabolites are found in high concentrations in the intestinal tract, where they are taken up by intestinal epithelial cells (IECs) and used as a substrate for oxidative production of adenosine triphosphate (ATP). In addition, these molecules act as a link between the microbiota and the immune system by modulating different aspects of IECs and the development, survival, and function of leukocytes. Furthermore, SCFAs have been shown to maintain intestinal homeostasis by protecting epithelial barrier integrity, promoting B-cell IgA production, and regulating T-cell differentiation.

Tests were performed on polypropylene microplates with 96 flat bottom holes (300 μL), and the absorbance determined in a microplate reader from Biotek, Synergy HT Multi-Mode. Reagents used were ethyl butyrate (99.0%) from Acros-Organics; ethyl acetate (99.5%), methyl propionate (99.5%), and acetone (99.5%) from Sigma-Aldrich and anhydrous monobasic and dibasic sodium phosphates (99.0%) from Vetec. Controls were butyric acid (99.5%) and acetic acid (99.8%) from Neon and propionic acid (99.0%) from Mallinckrodt. Bromothymol blue indicator was obtained from Synth and PDA (Potato Dextrose Agar) and PDB (Potato Dextrose Broth) culture media were obtained from Acumed.

Microbial strains present in the probiotic component of the synbiotic compositions of the present invention were screened for the production of organic acids using a gut modified medium with lactulose as the sole carbon source and bromocresol purple (0.5%) as an acid indicator that turns yellow under acidic conditions. The isolates that utilize lactulose to produce organic acids/short chain fatty acids and in turn acidify the medium are considered “hits.” Some strains are tested in liquid medium, while others are tested on a solid medium substrate.

The medium for measuring organic acid production had the composition according to Table 2.

TABLE 2

| Substance | Amount per liter | |
|---|---|---|
| Peptone | 2 | g |
| Yeast extract | 1 | g |
| KH₂PO₄ | 2 | g |
| MgSO₄•7 H₂O | 0.002 | g |
| NaCl | 0.08 | g |
| CaCl₂ | 8 | mg |
| FeSO₄•7 H₂O | 0.73 | mg |
| Hematin | 1.2 | mg |
| ATCC vitamin mix | 10 | mL |
| ATCC trace mineral mix | 10 | mL |
| Tween 80 | 0.5 | mL |
| L-cysteine | 0.5 | g |

| | |
|---|---|
| Agar | 20 g (for solid medium tests only) |
| Water | Balance |

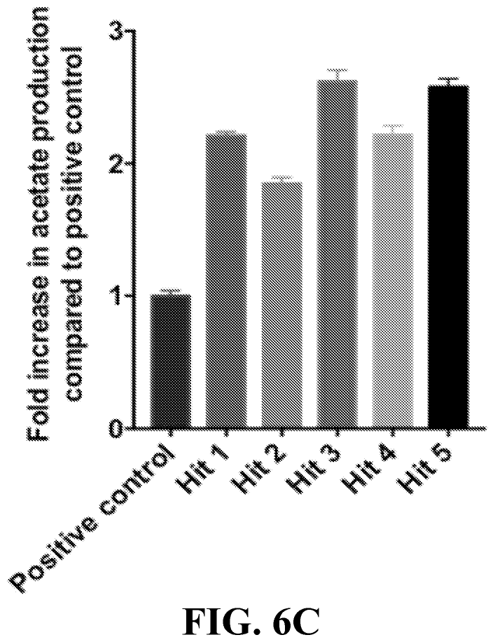

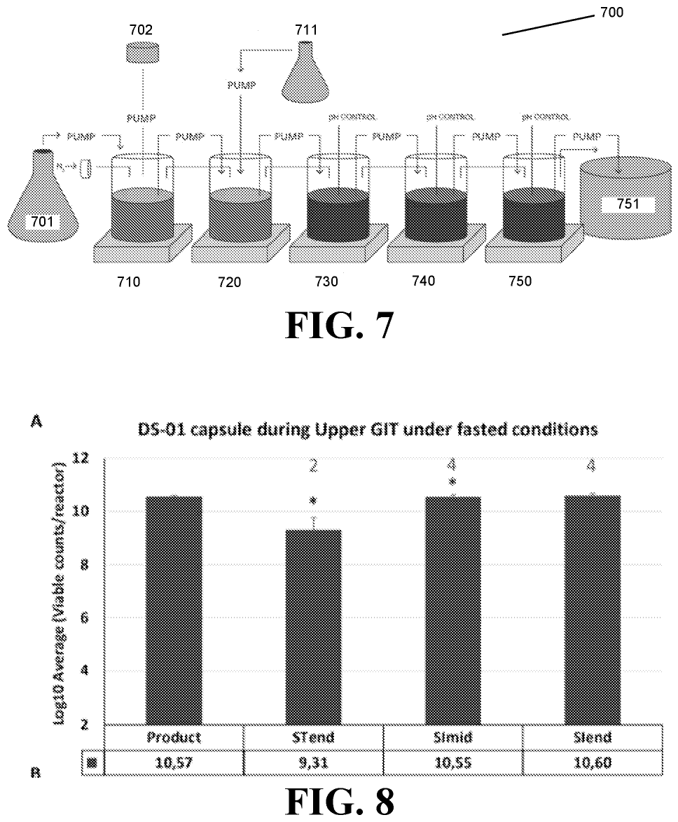

The production of organic acids was monitored by observing color changes in bromocresol purple to a yellow color after addition of the microbial strains. FIGS. 6A and 6B show the results for representative assays of the samples tested on liquid media and solid media, respectively. Visible light absorbance readings, illustrated in FIG. 7, provide quantitative measures of the amount of organic acid produced in each sample relative to the control.

As the results presented in FIGS. 6A, 6B, and 7 illustrate, administration of the synbiotic compositions of the present invention may increase the production of organic acids in the gastrointestinal tract. Thus, without wishing to be bound by any particular theory, it is believed that such administration may protect epithelial barrier integrity, promote B-cell IgA production, and/or regulate T-cell differentiation, and may thereby be effective to help maintain intestinal homeostasis.

### Example 5

### Gastrointestinal Survivability

To confirm the survival of the microbial consortia of the probiotic component of synbiotic compositions according to the present invention, hypromellose delivery capsules 100 according to FIG. 1A comprising the synbiotic composition were tested using a simulator of the human intestinal microbial ecosystem (SHIME) 700, illustrated in FIG. 7. Particularly, the SHIME 700 includes a stomach vessel 710, a small intestine vessel 720, an ascending colon vessel 730, a transverse colon vessel 740, and a descending colon vessel 750. The stomach vessel 710 receives and mixes the microbial consortia 701 and gastric acids 702 via pumps, as well as a supply of nitrogen gas. Output from the stomach vessel 710 is pumped to the small intestine vessel 720, where it is mixed with pancreatic juice 711 received via a pump. Output from the small intestine vessel 720 is pumped to the ascending colon vessel 730, which is pH-controlled to simulate the human ascending colon. Output from the ascending colon vessel 730 is pumped to the transverse colon vessel 740, which is pH-controlled to simulate the human transverse colon. Output from the transverse colon vessel 740 is pumped to the descending colon vessel 750, which is pH-controlled to simulate the human descending colon. Output from the descending colon vessel 750 is finally pumped to an effluent tank 751. The SHIME 700 thus recreates the physiological conditions and biological conditions (e.g. food uptake, peristalsis, digestive enzymes, pancreatic and bile acids, residence time, etc.) representative of the human gastrointestinal tract. Viability of the microorganisms was assessed using live/dead flow cytometry at various points throughout testing. Additionally, visual scoring of the capsules allowed observation of their disintegration behavior and possible targeted delivery of the probiotics: at each sampling point a visual inspection of the capsules was performed to study their dissolution behavior during passage through the different regions of the upper GIT, and at each sampling point capsules received a numerical score of 1 (capsule fully intact), 2 (capsule damaged but almost all product still in the capsule), 3 (capsule damaged and all product released), or 4 (capsule destroyed). Cytometry and visual inspection was carried out at four time points: before introduction of the capsules into the SHIME, at the end of residence in the stomach vessel 710 (approximately one hour after introduction), at an approximate midpoint of residence in the small intestine vessel 720 (approximately two hours after introduction), and at the end of residence in the small intestine vessel 720 (approximately three hours after introduction).

Referring now to FIG. 8, the results of testing of the delivery capsules 100 using the SHIME 700 are shown; the bars represent the base-10 logarithm of viable bacterial counts in the appropriate reactor, while the numerals above the bar indicate the qualitative 1-4 score obtained by visual inspection of the capsules. “Product,” “STend,” “SImid,” and “SIend” represent the first, second, third, and fourth timepoints, i.e. t=0, 1 hour, 2 hours, and 3 hours, respectively.

As illustrated in FIG. 8, after three hours of exposure to the conditions of the human stomach and small intestine, the capsules 100 were fully dissolved and/or destroyed, as indicated by the qualitative visual inspection score of 4 (capsule destroyed), but delivered a maximal release of living probiotic-remarkably, 100% of the starting dose of microbes (log-10.60 viable counts, as compared to a starting dose of log-10.57 viable counts) remained after this three-hour period. These results indicate that the delivery capsules of the present invention are effective to ensure viability of the full probiotic component of the synbiotic compositions of the present invention through to the end of the small intestine, and thus to release the full complement of microbes intact into the colon.

### Example 6

### Protective Effect Against Dysbiosis

This Example provides insight into the protective effects of the synbiotic compositions of the present invention against microbial dysbiosis caused by two common environmentally induced stressors, antibiotics and alcohol, by assessing return to baseline and overall microbial fermentation and metabolic activity in a transplanted human microbiota.

There is growing evidence that dietary habits, such as alcohol consumption or use of antibiotics, play a role in modulating the composition and metabolic activity of the human gut microbiota. In the United States, healthcare providers prescribe over 270 million antibiotic prescriptions each year. While antibiotics have transformed medicine and methods of treating life-threatening bacterial infection, broad spectrum antibiotics also induce disruption of resident gut microbial communities by altering both composition and function. This disruption of microbial community dynamics has been demonstrated at the taxonomic level, yet the extent of functional disruptions to microbial metabolic output and host cells remains understudied in humans.

The multiple layers of defence in the intestinal barrier, including physical, humoral and immunological components, can also be affected by alcohol. Animal and human studies have shown that alcohol consumption causes “leaky gut,” translocation of bacteria and microbial compounds across the intestinal basement membrane into the portal and systemic circulations. Additionally, alcohol consumption leads to intestinal bacterial dysbiosis and bacterial overgrowth in the small intestine in humans as well as in mouse models of alcohol consumption.

SHIME reactors mimicking the colonic environment were inoculated with fecal microbiota from a healthy donor, along with a nutritional source for the microbes and mucin-coated carriers to simulate the mucus environment. All test conditions were evaluated in triplicate.

Dysbiosis was induced by adding one of two different antimicrobial agents: antibiotics (50 μg/ml metronidazole+30 μg/ml ciprofloxacin) or alcohol (0.3 mL Grey Goose vodka/mL colon suspension, or 30% (v/v), corresponding with a 12% alcohol concentration). These doses were estimated at 10% of the oral antimicrobial input by assuming 90% absorption along the upper GIT, and were demonstrated in preliminary studies to induce substantial dysbiosis of the microbiota within the fermenter.

One full two-capsule dose (53 billion AFU) of the probiotic component of a synbiotic composition according to the present invention was added to the reactors containing antimicrobial-induced dysbiotic microbial communities. To simulate a ‘healthy’ control, a fermenter was inoculated with fecal microbiota from a healthy donor, with no antibiotics or alcohol added.

Microbial metabolism and activity were evaluated in each fermenter at baseline and after 6, 24, and 48 hours of incubation. The parameters evaluated were (1) change in overall fermentative activity as measured by pH (degree of acidification is a measure of the intensity of bacterial fermentative metabolism) and (2) changes in the concentrations of SCFA microbial metabolites, i.e. acetate, propionate, and butyrate (the pattern of SCFA production can be used to assess microbial carbohydrate and protein metabolism).

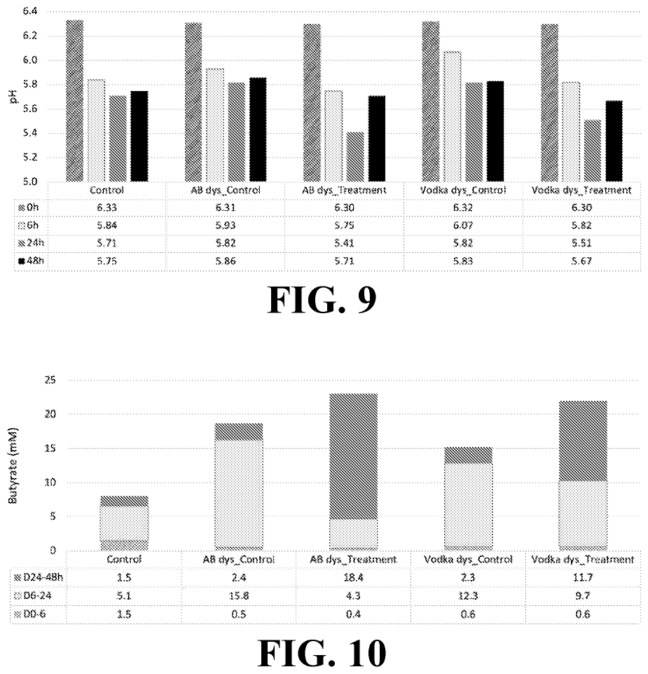

FIG. 9 illustrates the change in pH over 48 hours in the healthy control fermenter (“Control”), the antibiotic-induced dysbiotic control fermenter (“AB dys_Control”), the antibiotic-induced dysbiotic treatment fermenter (“AB dys_Treatment”), the alcohol-induced dysbiotic control fermenter (“Vodka dys_Control”), and the alcohol-induced dysbiotic treatment fermenter (“Vodka dys_Treatment”). The healthy control microbiota strongly reduced the pH of the fermenter, indicating high fermentative metabolic activity of the microbes. The dysbiosis control incubations (no synbiotic) resulted in less pronounced pH decreases than the healthy control incubation, reflecting reduced microbial fermentative metabolism. Addition of the synbiotic composition of the invention to the microbially-imbalanced communities resulted in stronger pH decreases than the respective control incubations, suggesting a stimulatory effect of the synbiotic composition on the fermentation processes in these microbial communities. The observed pH decreases were even stronger than the healthy control incubation. These results indicate that the synbiotic composition actively contributed to the fermentation process in two different models of induced dysbiosis, and as such stimulated the production of SCFA and/or lactate.

Total SCFA levels are reflective of the overall fermentation of test ingredients. The dysbiotic control incubations resulted in significantly lower total SCFA concentrations than the healthy control incubation. Synbiotic treatment stimulated total SCFA production in both dysbiotic communities. This resulted in similar or higher SCFA concentrations in synbiotic-treated dysbiotic fermenters as compared to the healthy controls by the end of incubation.

FIG. 10 illustrates the change in butyrate concentration over 48 hours in the healthy control fermenter (“Control”), the antibiotic-induced dysbiotic control fermenter (“AB dys_Control”), the antibiotic-induced dysbiotic treatment fermenter (“AB dys_Treatment”), the alcohol-induced dysbiotic control fermenter (“Vodka dys_Control”), and the alcohol-induced dysbiotic treatment fermenter (“Vodka dys_Treatment”). Butyrate is produced by members of the Clostridium genus and has a range of human health benefits. The dysbiotic control incubations produced significantly higher butyrate concentrations than the healthy control incubation. This mostly resulted from a higher production of butyrate between 6 hours and 24 hours and is likely explained by the fact that the luminal microbiota that primarily consists of acetate and propionate producers was highly affected by the dysbiotic agents, thereby resulting in preferential outgrowth of butyrate producers when the dysbiotic agents were gone. Synbiotic treatment increased butyrate production in both dysbiotic communities mostly during the last 24 hours. By the end of the incubation synbiotic-treated communities thus resulted in significantly higher butyrate concentrations than both the healthy control and the untreated dysbiotic incubations.

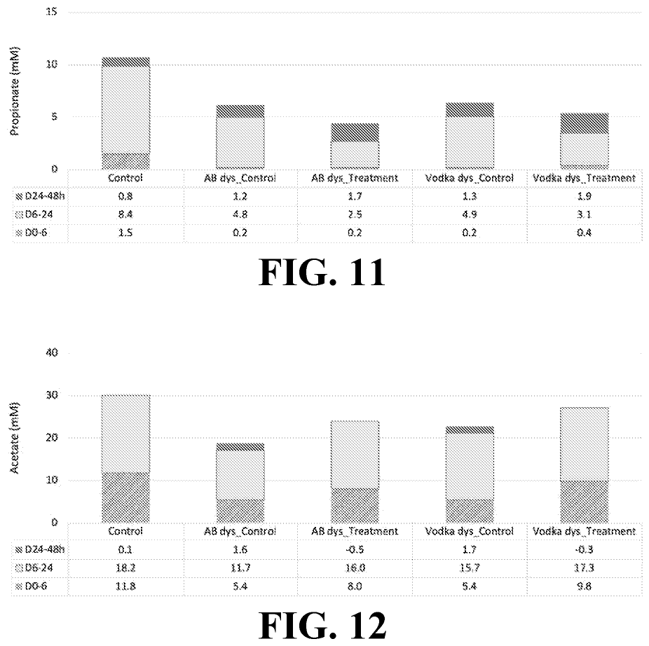

FIG. 11 illustrates the change in propionate concentration over 48 hours in the healthy control fermenter (“Control”), the antibiotic-induced dysbiotic control fermenter (“AB dys_Control”), the antibiotic-induced dysbiotic treatment fermenter (“AB dys_Treatment”), the alcohol-induced dysbiotic control fermenter (“Vodka dys_Control”), and the alcohol-induced dysbiotic treatment fermenter (“Vodka dys_Treatment”). Propionate is produced by a wide range of gut microbes, with the most abundant producers including Bacteroides species and Akkermansia muciniphila. The dysbiotic control incubations resulted in significantly lower propionate concentrations than the healthy controls. Synbiotic treatment further decreased propionate production in the first 24 hours, with an increase in the last 24 hours, in both dysbiotic communities. By the end of the incubation the treated communities thus resulted in lower propionate concentrations than the healthy control and dysbiotic controls. This can likely be explained by the highly specific stimulation of acetate and butyrate.

FIG. 12 illustrates the change in acetate concentration over 48 hours in the healthy control fermenter (“Control”), the antibiotic-induced dysbiotic control fermenter (“AB dys_Control”), the antibiotic-induced dysbiotic treatment fermenter (“AB dys_Treatment”), the alcohol-induced dysbiotic control fermenter (“Vodka dys_Control”), and the alcohol-induced dysbiotic treatment fermenter (“Vodka dys_Treatment”). Acetate is a primary metabolite produced in fermentation of prebiotic fibers. The dysbiotic control communities produced significantly lower acetate concentrations than the healthy controls. Synbiotic treatment stimulated acetate production in both dysbiotic communities. At the end of the study, acetate levels in synbiotic-treated dysbiotic incubations were significantly higher than before treatment, but still lower than that of healthy controls.

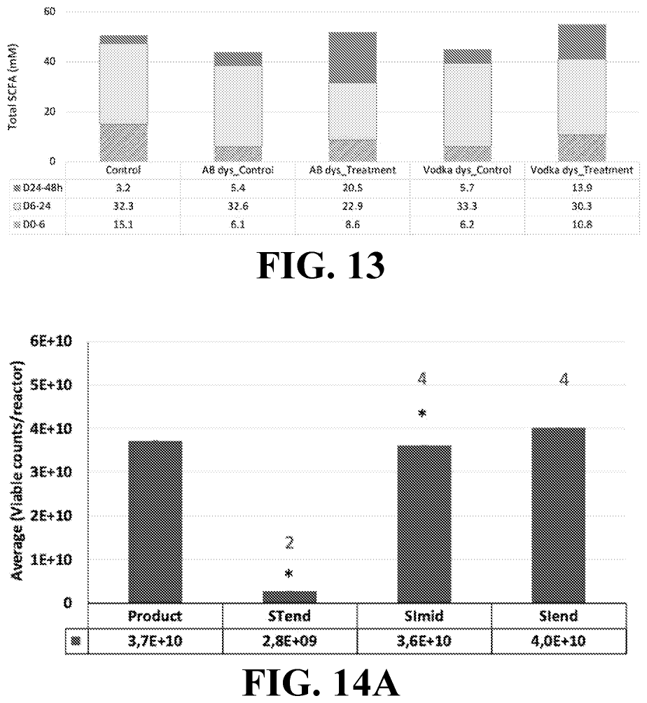

FIG. 13 illustrates the change in total SCFA concentration over 48 hours in the healthy control fermenter (“Control”), the antibiotic-induced dysbiotic control fermenter (“AB dys_Control”), the antibiotic-induced dysbiotic treatment fermenter (“AB dys_Treatment”), the alcohol-induced dysbiotic control fermenter (“Vodka dys_Control”), and the alcohol-induced dysbiotic treatment fermenter (“Vodka dys_Treatment”). The results of this Example indicate that incubation of synbiotic compositions of the present invention with the donor microbiota stimulated short chain fatty acid production, most interestingly butyrate, upon both antibiotic-induced and alcohol-induced dysbiosis. The butyrogenic effect of synbiotic treatment was so strong that butyrate production of the dysbiotic communities largely exceeded those of the healthy control communities.

### Example 7

### Comparative Gastrointestinal Survivability

The procedure of Example 5 was repeated using both a synbiotic composition capsule according to the present invention and seventeen commercially available conventional probiotic products, listed in Table 3 below.

TABLE 3

| # | Product |
|---|---|
| 1 | Present invention |
| 2 | Align Probiotics |
| 3 | Culturelle Daily Probiotic |
| 4 | VSL#3 |
| 5 | Hyperbiotics |
| 6 | Metagenics UltraFlora |
| 7 | Thorne FloraMend Prime |
| 8 | Renew Life |
| 9 | Garden of Life Doctor's Formula |
| 10 | Yakult |
| 11 | Lifespace |
| 12 | Duolac |
| 13 | Jarrow |
| 14 | Megasporebiotics |
| 15 | Nature's Bounty Ultra Strength Probiotic 10 |
| 16 | TruBiotics Daily Probiotic |
| 17 | Blackmores Probiotics+ |
| 18 | Swisse Daily Digestive Probiotic |

At the beginning of the stomach incubation, one capsule was administered per reactor, except for products 10 and 12. Capsules of all products excepts products 10 and 12 were mounted in a capsule sinker. To investigate the Yakult product (product 10), one dose (65 mL) was administered at the beginning of the gastric phase to a concentrated gastric suspension to obtain the same start volume as the other incubations. The Duolac product (product 12) was investigated by addition of one dose (2.5 g) at the beginning of the stomach incubation. All experiments were performed in biological triplicate to account for biological variability.

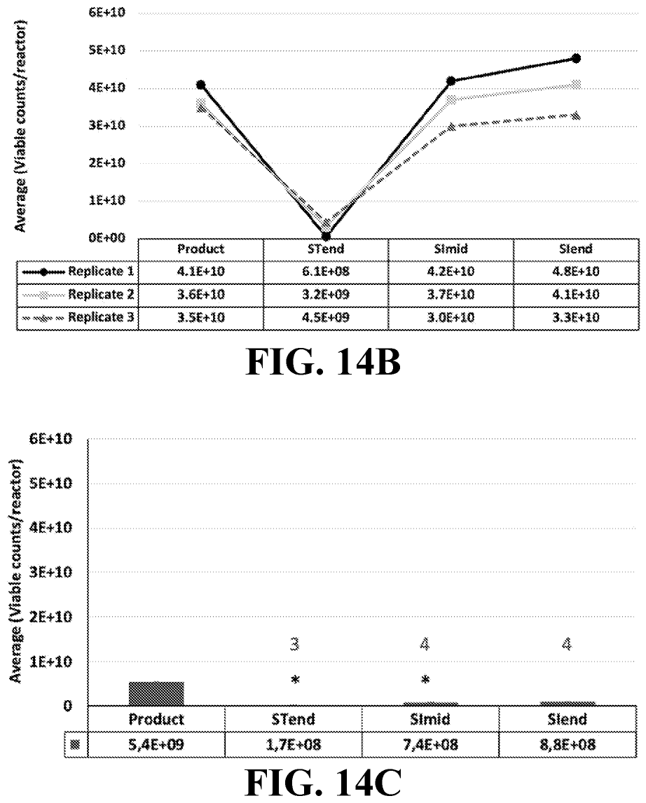

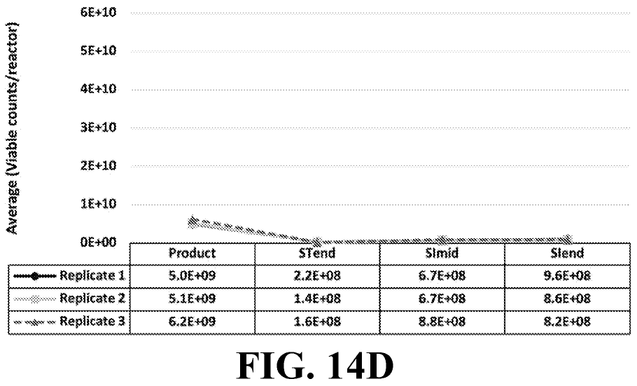

The hypromellose synbiotic composition capsules 100 according to the present invention released at least a fraction of the capsule content (qualitative score of 2), resulting in an average of 2.8·10⁹ viable bacteria at the end of the gastric phase. The capsules 100 then achieved complete release (qualitative score of 4) after 1.5 hours of incubation in the small intestine vessel 720. As illustrated in FIGS. 14A and 14B, viability of the released bacterial cells approached and remained near 100%. Prolonged small intestinal incubation did not affect viability, indicated by complete survival of the administered probiotics at the end of the small intestinal phase. Overall, these results indicate that the individual cells are able to survive co-incubation in the consortium in isolation and during exposure to stomach acid, proteases, lipases, and bile salts encountered during gastrointestinal transit in all replicates, resulting in the delivery of, on average 4.0·10¹⁰ viable bacterial cells to the colon after passage through the upper gastrointestinal tract under fasted conditions.

By contrast, the conventional probiotic products exhibited significantly decreased delivery of viable bacterial cells at the end of the small intestine compared to the synbiotic composition capsules 100 according to the present invention, as illustrated in Table 4. “Naked” products (i.e. products not contained within a capsule), such as Yakult and Duolac, resulted in the lowest survival (0.7% and 0.3%, respectively). The Swisse Daily Digestive Probiotic also resulted in a low survival of 0.8% due to fast release of the capsule contents in the intestinal lumen. The best performing conventional probiotic products-Metagenics UltraFlora, Jarrow, and Blackmore Probiotics-delivered only between about 34% and 57% of viable bacterial cells to the colon, compared to the approximately 100% of the synbiotic composition capsules 100 according to the present invention.

TABLE 4

| | | % delivery of viable |
|---|---|---|
| | | cells at end of |
| # | Product | small intestine |
| 1 | Present invention | 108.2% ± 20.1% |
| 2 | Align Probiotics | 16.3% ± 1.4% |
| 3 | Culturelle Daily Probiotic | 8.5% ± 4.1% |
| 4 | VSL#3 | 21.4% ± 1.1% |
| 5 | Hyperbiotics | 32.1% ± 12.1% |
| 6 | Metagenics UltraFlora | 56.2% ± 6.7% |
| 7 | Thorne FloraMend Prime | 8.5% ± 2.1% |
| 8 | Renew Life | 23.6% ± 8.8% |
| 9 | Garden of Life Doctor's Formula | 33.6% ± 5.6% |
| 10 | Yakult | 0.14% ± 0.06% |
| 11 | Lifespace | 19.8% ± 9.5% |
| 12 | Duolac | 0.33% ± 0.37% |
| 13 | Jarrow | 39.1% ± 1.2% |
| 14 | Megasporebiotics | 2.0% ± 0.1% |
| 15 | Nature's Bounty Ultra Strength Probiotic 10 | 9.3% ± 8.1% |
| 16 | TruBiotics Daily Probiotic | 1.9% ± 0.5% |
| 17 | Blackmores Probiotics+ | 34.8% ± 2.3% |
| 18 | Swisse Daily Digestive Probiotic | 0.75% ± 1.21% |

Results obtained from the Align© Probiotic Capsules are illustrative of drawbacks observed for many other conventional probiotic products. Throughout the gastric phase, the Align© Probiotic capsules released a significant fraction of the encapsulated bacteria. As such, a complete dissolution of the capsules (qualitative score of 4) was obtained after 1.5 hours of incubation in the small intestine vessel 720. As illustrated in FIGS. 14C and 14D, a total of log-8.87 viable bacteria were detected after passage through the upper GIT, indicating a reduction of approximately log-0.85 after 1.5 hours of small intestinal incubation compared to the composition of the present invention. Without wishing to be bound by any particular theory, this decrease is likely due to the early gastric release, in which the microbial strains encountered the low environmental pH and/or high concentrations of microbe-toxic bile salts at the beginning of the small intestinal incubation. Prolonged small intestinal incubation did not affect viability further. Overall, only 16% of the viable bacteria were delivered at the end of the small intestinal phase, which corresponds with an average of log-8.94 viable bacterial cells. Thus, the synbiotic composition and hypromellose delivery capsule 100 of the present invention provided significantly improved delivery of viable bacterial cells at the end of the small intestine (i.e. upon entry into the colon) compared to the conventional Align® probiotic product.

### Example 8

### Histamine Metabolism

Histamine is an important biogenic amine in human health, as it modulates a variety of processes ranging from muscle contraction to immunomodulation. Excess histamine, however, can cause histamine intolerance, which negatively impacts health and general wellbeing. While histamine intolerance is typically attributable to a genetic diamine oxidase deficiency, growing evidence supports microbial involvement, with emergent concern arising over the consumption of live microorganisms in the form of fermented food items or probiotics. This Example tested the in vitro production of these compounds. Microbial strains present in the probiotic component of the synbiotic compositions of the present invention, as well as various controls strains, were grown in both aerobic and anaerobic environments and subsequently analyzed for the presence of histamine and both lactate isoforms.

A single hypromellose delivery capsule according to the present invention was opened and the contents aseptically added to a 50 mL conical tube containing 45 mL De Man, Rogosa and Sharpe (MRS) liquid broth medium (Sigma Aldrich). Each strain of the synbiotic compositions of the present invention is known to grow in this medium. The reason for testing all of strains together was to simulate what would occur upon human consumption. The tubes were vortexed for 30 seconds to ensure the contents of the capsules were dissolved and equally dispersed throughout the medium. Samples were then incubated at 37° C. under stationary conditions in an aerobic or anaerobic environment for 96 h to assess histamine production under varying levels of oxygen.

Lactobacillus reuteri ATCC 23272, used as a positive control, was streak-plated from frozen stock onto MRS agar and incubated anaerobically at 37° C. overnight. A single colony was selected and inoculated for 12 hours at 37° C. in MRS broth under anaerobic conditions. Subsequently, the overnight cultures were subcultured (1:225 dilution) into fresh MRS broth medium. Cultures were then incubated at 37° C. under anaerobic conditions for 96 h prior to histamine analysis.

A competitive enzyme-linked immunosorbent assay (ELISA) was used to quantify the concentration (ng/mL) of histamine in each sample. Aliquots of 1 mL from each sample were centrifuged at 1000 g for 20 minutes at 4° C. Subsequently, the supernatant was used to quantify histamine following manufacturers' instructions (Histamine ELISA Kit; E-EL-0032; Elabscience).

Total protein contents in the supernatants of tested cultures were determined using a Pierce BCA Protein Assay Kit (Thermo Scientific) following the manufacturer's instructions. Briefly, 25 L of each sample (aliquoted from the same supernatant extracted during previous lactate quantification steps) was added to 200 L of the working reagent, mixed thoroughly, and then incubated under stationary conditions in the dark at 37° C. for 30 minutes. Subsequently, colorimetric detection and quantitation of total protein was determined by measuring optical density at 562 nm using a BioTek PowerWave HT microplate reader (BioSPX).

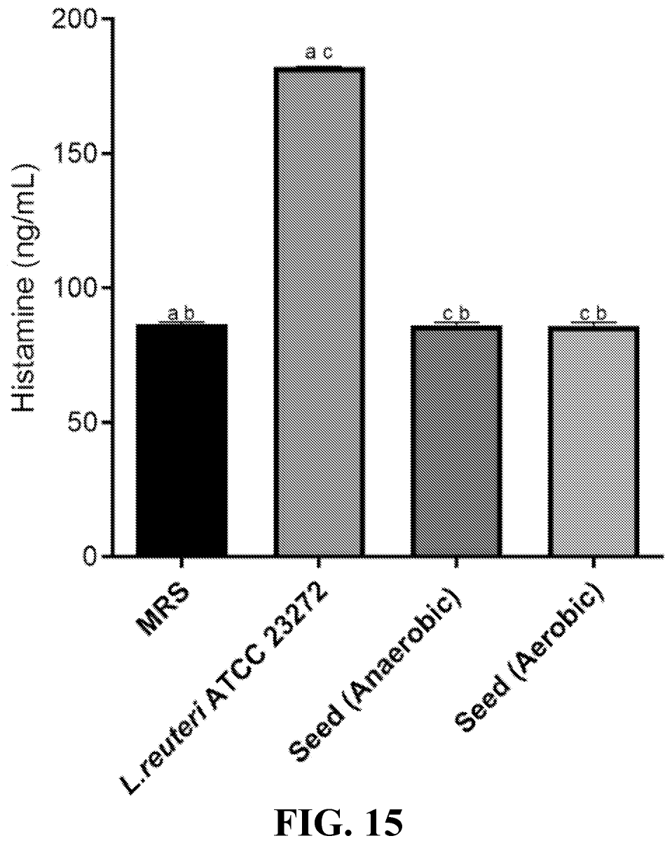

Growth of the strains of the synbiotic compositions of the present invention, under anaerobic conditions, yielded a mean of 85.99±1.23 ng/mL histamine (SD 85.76±1.42 ng/mL). The uninoculated vehicle (MRS media alone) was also analyzed and found to contain 86.44±0.92 ng/mL of histamine, as illustrated by the bar labeled “MRS” in FIG. 15. No detectable differences in histamine production were observed for cultures grown aerobically (the bar labeled “Seed (Aerobic)” in FIG. 15) or anaerobically (the bar labeled “Seed (Anaerobic)” in FIG. 15), in comparison to the vehicle control (ANOVA, P<0.05). L. reuteri ATCC 23272, a strain previously shown to produce histamine, demonstrated significantly higher histamine content in its culture supernatant compared with the vehicle control (ANOVA, P<0.05), as illustrated in FIG. 15.

### Example 9

### Comparative Shelf Stability and Water Activity

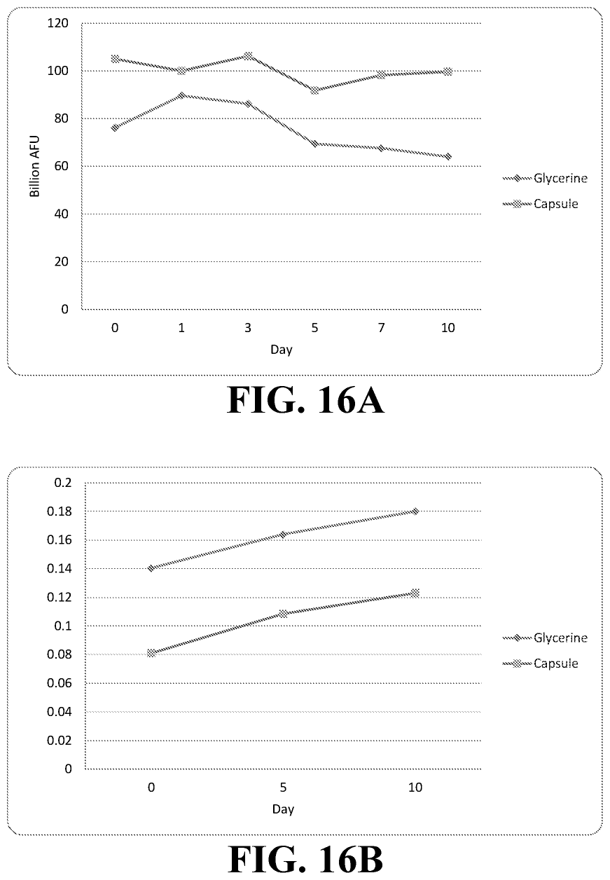

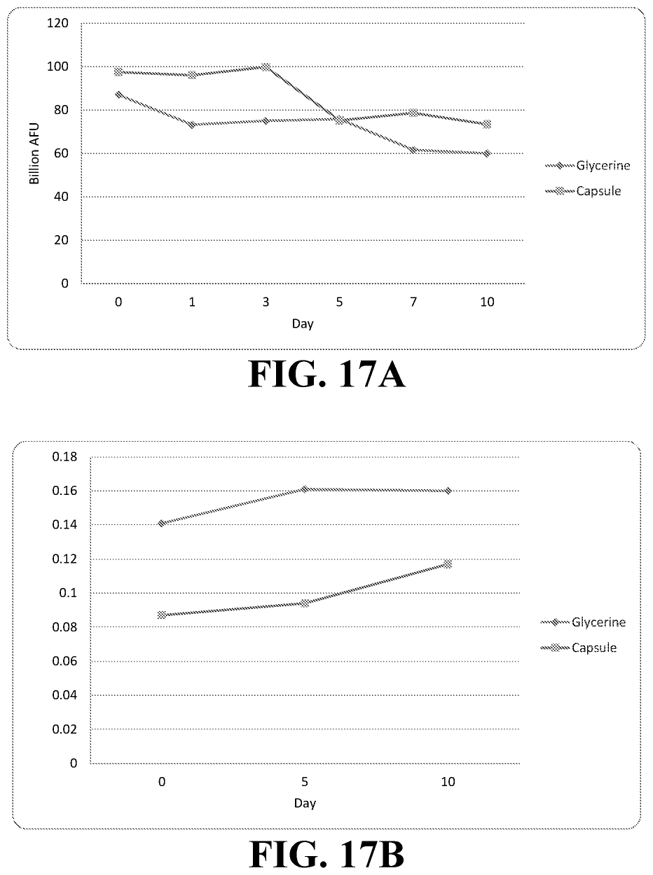

The probiotic component of the present invention was incorporated into both hypromellose delivery capsules 100 and a liquid glycerol solvent. Samples of each of these delivery vehicles were held at two different temperature and humidity conditions for ten days: a 25±2° C., 50%±5% relative humidity condition, and a 35±2° C., 75%±5% relative humidity condition. The number of living cells in each sample was determined at 0, 1, 3, 5, 7, and 10 days, and the water activity of the composition was determined at 0, 5, and 10 days. The number of living cells and water activity under the low-temperature/low-humidity condition are illustrated in FIGS. 16A and 16B, respectively, and the number of living cells and water activity under the high-temperature/high-humidity condition are illustrated in FIGS. 17A and 17B, respectively.

As FIGS. 16A and 17A illustrate, the delivery capsules 100 of the present invention are superior to a liquid glycerol solvent for maintaining a substantial fraction of original cells as viable under normal storage conditions over periods of at least about ten days. This feature is crucial for ensuring potency and compliance with labeling requirements. Likewise, as FIGS. 16B and 17B illustrate, the delivery capsules 100 of the present invention maintain lower water activity than a liquid glycerol solvent over a period of at least about ten days. Decreased water activity is desirable for a number of reasons, particularly for mitigating contamination of the probiotic composition with extrinsic microbes from the environment.

### Example 10

### Accelerated Stability Testing

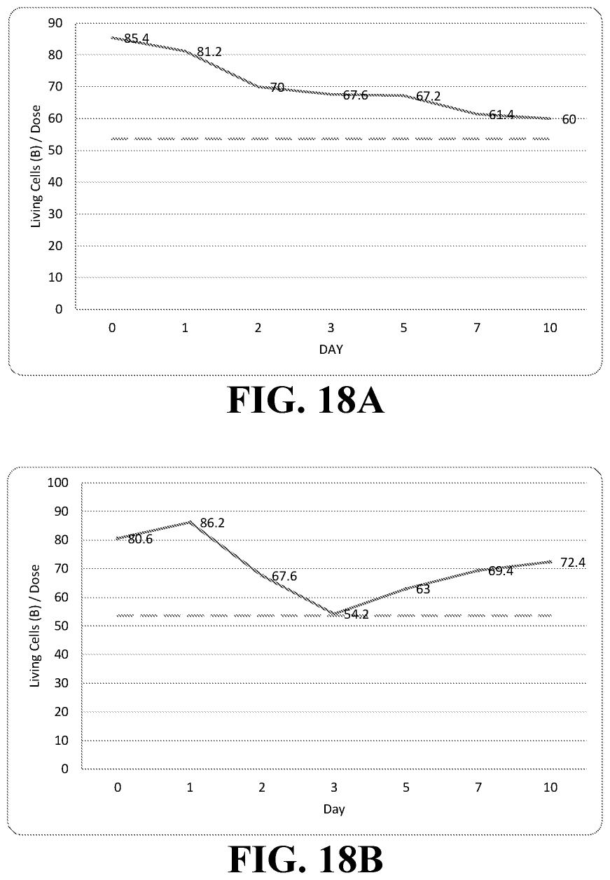

Hypromellose delivery capsules 100 comprising the synbiotic composition of the present invention were packaged in both a probiotic storage jar and a probiotic storage pouch. The jar was maintained at 49±2° C. and 50%±5% relative humidity for ten days, and the pouch was maintained at 38±2° C. and 50%±5% relative humidity for ten days. The number of living cells in one dose (two capsules) was measured at 0, 1, 2, 3, 5, 7, and 10 days; the capsules were adapted to deliver one dose (53.6 billion AFU of the probiotic component) of the synbiotic composition. The number of living cells from the jar capsules and pouch capsules are illustrated in FIGS. 18A and 18B, respectively (the cell count target of 53.6 billion AFU is illustrated as a dashed line).

As FIGS. 18A and 18B illustrate, the delivery capsules 100 of the present invention are effective to maintain a sufficient fraction of original cells as viable under accelerated degradation conditions over a period of at least about ten days, whether stored in jars or in pourches. This feature is crucial for ensuring potency and compliance with labeling requirements.

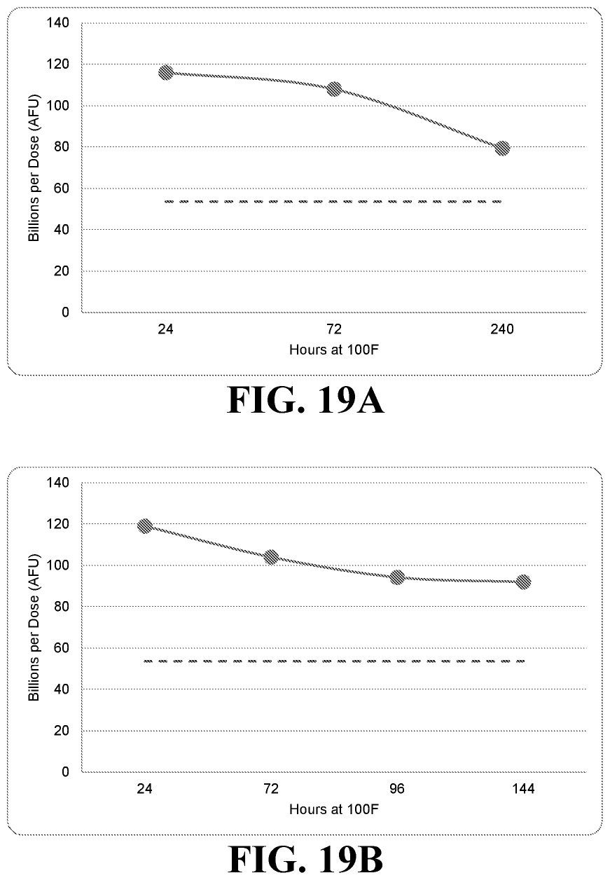

This procedure was repeated, with both the jar and the pouch held at 100° F.; the number of living cells per dose was measured at 1, 3, and 10 days for the jar capsules and 1, 3, 4, and 6 days for the pouch capsules. The results are illustrated in FIGS. 19A and 19B, respectively, again indicating successful maintenance of an appropriate count of cells as viable.

### Example 11

### Real-Time Stability Testing

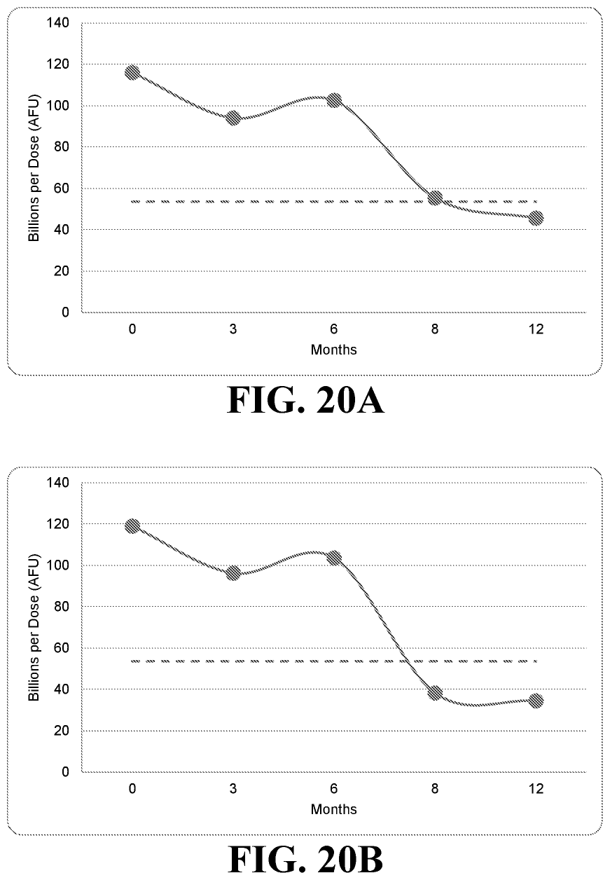

The procedure of Example 10 was repeated, except that the jar and pouch were maintained at normal room temperature and humidity rather than elevated temperature to more closely simulate long-term storage conditions. The number of living cells per dose (two capsules) was measured at 0, 3, 6, 8, and 12 months; the results for the jar and the pouch are given in FIGS. 20A and 20B, respectively (target cell count again illustrated as a dashed line). These results again illustrate that the delivery capsules 100 of the present invention, whether stored in jars or in pouches, are effective to maintain a sufficient fraction of original cells as viable under typical degradation conditions over a period of at least about six months, and in jars over a period of at least about eight months.

The invention illustratively disclosed herein suitably may be practiced in the absence of any element which is not specifically disclosed herein. It is apparent to those skilled in the art, however, that many changes, variations, modifications, other uses, and applications of the invention are possible, and also changes, variations, modifications, other uses, and applications which do not depart from the spirit and scope of the invention are deemed to be covered by the invention, which is limited only by the claims which follow.

The foregoing discussion of the invention has been presented for purposes of illustration and description. The foregoing is not intended to limit the invention to the form or forms disclosed herein. In the foregoing Detailed Description of the Invention, for example, various features of the invention are grouped together in one or more embodiments for the purpose of streamlining the disclosure. The features of the embodiments of the invention may be combined in alternate embodiments other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description of the Invention, with each claim standing on its own as a separate preferred embodiment of the invention.

Moreover, though the description of the invention has included description of one or more embodiments and certain variations and modifications, other variations, combinations, and modifications are within the scope of the invention, e.g. as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, whether or not such alternate, interchangeable, and/or equivalent structures, functions, ranges, or steps are disclosed herein, and without intending to publicly dedicate any patentable subject matter.

1. A swallowable capsule for enteral administration of a synbiotic composition, comprising:
an inner capsule, comprising a probiotic component of the synbiotic composition, the probiotic component comprising a consortium of microbial strains; and
an outer capsule, surrounding and enclosing the inner capsule, comprising a prebiotic component of the synbiotic composition,
wherein the outer capsule is configured to be substantially completely destroyed or dissolved after three hours in the environment of the human stomach and small intestine,
wherein the inner and outer capsules are configured such that a proportion of cells in the consortium of microbial strains that remain viable after three hours in the environment of the human stomach and small intestine is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, and
wherein the inner capsule is configured, upon entry into the colon of a human subject to whom the swallowable capsule is administered, to release at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of viable cells of the consortium of microbial strains into the colon.

2. The capsule of claim 1, comprising the prebiotic component in an amount of from about 1 mg to about 400 mg, or from about 25 mg to about 375 mg, or from about 50 mg to about 350 mg, or from about 75 mg to about 325 mg, or from about 100 mg to about 300 mg, or from about 125 mg to about 275 mg, or from about 150 mg to about 250 mg, or from about 175 mg to about 225 mg, or about 200 mg.

3. The capsule of claim 1, comprising the consortium of microbial strains in an amount of from about 62.5 million AFU to about 312.5 billion AFU, from about 625 million AFU to about 250 billion AFU, from about 1.25 billion AFU to about 125 billion AFU, from about 6.25 billion AFU to about 62.5 billion AFU, from about 12.5 billion AFU to about 50 billion AFU, from about 18.75 billion AFU to about 37.5 billion, or from about 25 billion AFU to about 31.25 billion AFU.

4. The capsule of claim 1, wherein the inner capsule further comprises a pharmaceutically acceptable vehicle for the probiotic component.

5. The capsule of claim 1, wherein the outer capsule further comprises a pharmaceutically acceptable vehicle for the prebiotic component.

6. The capsule of claim 1, substantially free of any liquid vehicle or solvent.

7. The capsule of claim 1, wherein the prebiotic component comprises at least one compound that can be converted, by a microbial strain present in the healthy human gut microbiota, into a bioactive metabolite.

8. The capsule of claim 7, wherein the at least one compound that can be converted, by a microbial strain present in the healthy human gut microbiota, into a bioactive metabolite comprises at least one punicalagin.

9. The capsule of claim 8, wherein the at least one punicalagin is derived or extracted from at least one pomegranate.

10. The capsule of claim 9, wherein the prebiotic component further comprises at least one additional compound derived or extracted from at least one pomegranate.

11. The capsule of claim 9, wherein the prebiotic component consists essentially of a polyphenolic pomegranate derivative or extract comprising the at least one punicalagin.

12. The capsule of claim 8, wherein the at least one punicalagin is capable of being metabolized, by at least one bacterial strain known to inhabit the human gastrointestinal tract, into a urolithin.

13. The capsule of claim 12, wherein the urolithin is urolithin-A.

14. The capsule of claim 8, wherein the at least one punicalagin is capable of being metabolized, by at least one microbial strain of the consortium of the probiotic component, into a urolithin.

15. The capsule of claim 14, wherein the urolithin is urolithin-A.

16. The capsule of claim 1, wherein the consortium of microbial strains comprises at least two of:
(i) one or more digestive outcome-, gastrointestinal outcome-, or gut barrier function-improving microbial strains selected from the group consisting of Bifidobacterium breve SD-BR3-IT, Lactiplantibacillus plantarum SD-LP1-IT, Bifidobacterium longum SD-BB536-JP, Bifidobacterium infantis SD-M63-JP, Lacticaseibacillus rhamnosus HRVD113-US, Bifidobacterium lactis HRVD524-US (B1-04), Bifidobacterium breve HRVD521-US, Lacticaseibacillus casei HRVD300-US, Bifidobacterium longum HRVD90b-US, Bifidobacterium lactis SD150-BE, Lacticaseibacillus rhamnosus SD-GG-BE, Limosilactobacillus reuteri RD830-FR, Lactobacillus crispatus SD-LCR01-IT, Limosilactobacillus fermentum SD-LF8-IT, Bifidobacterium lactis SD-BS5-IT, and Lacticaseibacillus rhamnosus SD-LR6-IT;
(ii) one or more dermatological outcome-improving microbial strains selected from the group consisting of Ligilactobacillus salivarius SD-LS1-IT, Bifidobacterium longum SD-CECT7347-SP, Lacticaseibacillus casei SD-CECT9104-SP, and Bifidobacterium lactis SD-CECT8145-SP;
(iii) one or more cardiovascular outcome-improving microbial strains selected from the group consisting of Lactiplantibacillus plantarum SD-LPLDL-UK and Bifidobacterium lactis SD-MB2409-IT; and
(iv) one or more micronutrient-synthesizing microbial strains selected from the group consisting of Limosilactobacillus reuteri SD-LRE2-IT and Bifidobacterium adolescentis SD-BA5-IT.

17. The capsule of claim 16, wherein the consortium comprises at least three of (i) through (iv).

18. The capsule of claim 17, wherein the consortium comprises all four of (i) through (iv).

19. The capsule of claim 16, wherein the consortium comprises at least two of the digestive outcome-, gastrointestinal outcome-, or gut barrier function-improving microbial strains of (i).

20. The capsule of claim 16, wherein the consortium comprises all of the digestive outcome-, gastrointestinal outcome-, or gut barrier function-improving microbial strains of (i), all of the dermatological outcome-improving microbial strains of (ii), all of the cardiovascular outcome-improving strains of (iii), and all of the micronutrient-synthesizing strains of (iv).

21. The capsule of claim 16, wherein the consortium consists essentially of all of the digestive outcome-, gastrointestinal outcome-, or gut barrier function-improving microbial strains of (i), all of the dermatological outcome-improving microbial strains of (ii), all of the cardiovascular outcome-improving strains of (iii), and all of the micronutrient-synthesizing strains of (iv).
